(19) United States
(12) Patent Application Publication
(10) Pub. No.: US 2026/0174550 A1
(43) Pub. Date: Jun. 25, 2026

(54) Artificial Heart Valve

(71) Applicant: The David J Wheatley Discretionary Trust, Glasgow (GB)

(72) Inventors: David J. Wheatley, London (GB); Iain Wiliam Stewart, London (GB); James Clark St Clair Sean McKee, London (GB)

(21) Appl. No.: 19/126,082

(22) PCT Filed: Oct. 31, 2023
(86) PCT No.: PCT/GB2023/052843

§ 371 Date: Apr. 30, 2025

(30) Foreign Application Priority Data
Oct. 31, 2022 (GB) 2216084.0

(51) Int. Cl. A61F 2/24

(52) U.S. Cl. CPC A61F 2/2415; A61F 2/2409; A61F 2/243; A61F 2220/0075

(57) ABSTRACT

An artificial heart valve comprises a frame, a plurality of flexible leaflets attached to the frame, and a plurality of support members, each support member being attached to a corresponding leaflet. The leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction. The support members are movable with the leaflets relative to the frame. When the leaflets are in the closed configuration and exposed to a back pressure having at least a component in the backward direction, a force is exerted on the leaflets in the backward direction, and the support members at least partially prevent or resist deformation of the leaflets. A leaflet arrangement for an artificial heart valve and a method of manufacturing an artificial heart valve are also disclosed.

### FIELD

The present disclosure relates to an artificial heart valve, a leaflet arrangement for an artificial heart valve and to a method of manufacturing an artificial heart valve and, in particular though not exclusively, to a synthetic heart valve, a leaflet arrangement for a synthetic heart valve and to a method of manufacturing a synthetic heart valve.

### BACKGROUND

Referring initially to FIG. 1A, there is shown a known artificial heart valve generally designated 102 comprising three flexible leaflets 104 attached to a frame 106. The leaflets 104 may comprise, or be formed from, polyurethane. The frame 106 may comprise, or be formed from, stainless steel.

FIG. 1B shows the frame 106 in isolation from the leaflets 104. As may be appreciated from FIG. 1B, the frame 106 comprises a generally annular base 108 defining an aperture 109 and three posts 110 extending from the base 108. The aperture 109 defines an axis 111. The posts 110 are generally straight and extend from the base 108 in a direction which is generally parallel to the axis 111. Each post 110 defines an aperture in the form of a slit 140 for attachment of corresponding side portions of two of the leaflets 104 to the post 110.

Referring back to FIG. 1A, a base portion 112 of each leaflet 104 is outwardly convex relative to the axis 111 and extends partially around, and is attached to, the annular base 108 of the frame 106 along a corresponding base line of attachment. Side portions 114 of each leaflet 104 are attached to corresponding first and second adjacent posts 110 of the frame 106 along corresponding side lines of attachment. Each leaflet 104 has a free edge 115 which extends between the corresponding first and second adjacent posts 110 of the frame 106 opposite the base 108 of the frame 106. The free edge 115 of each leaflet 104 is free to move relative to the frame 106 in response to a difference in pressure between an inflow side of the leaflets 104 and an outflow side of the leaflets 104.

FIG. 2 shows a contour map of the leaflets 104 in their natural or as-formed configuration in which contours 1-11 represent constant height contours increasing in distance from the base 108 of the frame 106 such that contour 1 coincides with the base 108 of the frame of a leaflet 106 and contour 11 represents a boundary 116 of a co-aptation region 117 which extends from the free edge 115 of the leaflet to the boundary 116 of the co-aptation region 117. As may be appreciated from the contours shown in FIG. 2, each leaflet 104 comprises an outwardly convex region 121 and an outwardly concave region 122, wherein the corresponding outwardly convex and outwardly concave regions 121, 122 meet along a corresponding demarcation line 124. Moreover, as may be appreciated from the following description, the outwardly convex region 121 may comprise a region of an outer surface of a corresponding cone such as a right cone, an oblique cone or an elliptic cone. Similarly, as may be appreciated from the following description, the outwardly concave region 122 may comprise a region of an inner surface of a corresponding cone such as a right cone, an oblique cone or an elliptic cone.

As shown in FIG. 2, each leaflet 104 comprises a plurality of transverse cross-sections, wherein each transverse cross-section is taken through said leaflet 104 in a corresponding transverse plane which is transverse to the axis 111 and wherein each transverse cross-section extends along a corresponding curved line in the form of one of the contours 1-11 shown in FIG. 2. Each transverse plane extends through the outwardly convex region 121, the outwardly concave region 122, and the demarcation line 124 of said leaflet 104 so as to define a corresponding outwardly convex portion 119 of the corresponding transverse cross-section, a corresponding outwardly concave portion 120 of the corresponding transverse cross-section, and a corresponding point of inflection 118 of the corresponding transverse cross-section respectively. The corresponding outwardly convex portion 119 is outwardly convex relative to the axis 111, the corresponding outwardly concave portion 120 is outwardly concave relative to the axis 111, and the corresponding point of inflection 118 is located at a corresponding junction between the corresponding outwardly convex portion 119 and the corresponding outwardly concave portion 120 so that each transverse cross-section has an “S-shape” when viewed from an outflow direction. The corresponding outwardly convex portion 119 extends from a corresponding first post 110 to the corresponding point of inflection 118, and the corresponding outwardly concave portion 120 extends from the corresponding second post 110 to the corresponding point of inflection 118. Since each leaflet 104 is attached along a generally straight side line of attachment to a post 110 of the frame 106, the contours 1-11 shown in FIG. 2 meet in the vicinity of each post 110 of the frame 106.

In addition, as may be appreciated from FIG. 2, a first one of the transverse cross-sections taken through any one of the leaflets 104 on a first one of the transverse planes comprises a first outwardly convex portion, a first outwardly concave portion, and a first point of inflection, and a second one of the transverse cross-sections taken through any one of the leaflets on a second one of the transverse planes comprises a second outwardly convex portion, a second outwardly concave portion, and a second point of inflection, wherein the first one of the transverse planes is located closer to the base 108 of the frame 106 than the second one of the transverse planes and wherein the first outwardly convex portion is longer than the second outwardly convex portion and the first outwardly concave portion is shorter than the second outwardly concave portion.

From the foregoing description, it will be appreciated that each transverse cross-section through each leaflet 104 is longer than the base portion 112 of said leaflet 104 which is attached to the base 108 of the frame 106. Similarly, the free edge 115 of each leaflet 104 is longer than the corresponding base portion 112 of said leaflet 104.

Moreover, the point of inflection 118 corresponding to the free edge 115 is located half-way along, or substantially half-way along, the free edge 115 between the corresponding first and second adjacent posts 110. Furthermore, the co-aptation region 117 of each leaflet 104 comprises a plurality of corresponding generally identical transverse cross-sections. Consequently, the demarcation line 124 comprises first and second linear portions, wherein the first linear portion extends from the base 108 of the frame 106 to the point of inflection 118 of the transverse cross-section at the boundary 116 of the co-aptation region 117 and the second linear portion extends from the point of inflection 118 of the transverse cross-section at the boundary 116 of the co-aptation region 117 to the point of inflection 118 of the free edge 115.

In use, when the leaflets 104 are configured in the closed configuration so that the free edges 115 of the leaflets 104 extend along the dashed paths denoted “C” in FIG. 3 and the leaflets 104 are exposed to a back pressure acting in a backward direction on an outflow side of the leaflets 104, the radially inner surfaces of the co-aptation regions 117 of the leaflets 104 engage one another so as to reduce or prevent blood flow between the leaflets 104 in the backward direction. When the pressure exerted on the flexible leaflets 104 from an inflow side of the leaflets 104 sufficiently exceeds that exerted from the outflow side, the co-aptation regions 117 of the leaflets 104 disengage and the leaflets 104 move radially outwardly until the free edges 115 adopt an open configuration denoted “O” in FIG. 3 to create an outflow orifice 132, the maximum size of which can be varied by the design of the S-shape that determines the length of the free edges 115 of the leaflets 104. In FIG. 3, the contours 3, 5, 7 and 9 represent constant height contours of a leaflet 104 in the open configuration “O” i.e. transverse cross-sections of the leaflet 104 in the open configuration “O”. The free edges 115 of the leaflets 104 may adopt a natural or default configuration denoted “F” when no pressure differential exists across the leaflets 104, wherein the natural or default configuration denoted “F” is intermediate the closed configuration denoted “C” and the open configuration denoted “O”.

With reference to FIG. 3, as each leaflet 104 moves from its closed configuration “C” towards its open configuration “O” between two adjacent posts 110, the outwardly convex and concave regions 121, 122 of the leaflet 104 swing or pivot about their respective side lines of attachment at the corresponding posts 110. Moreover, movement of each leaflet 104 from its closed configuration “C” towards its open configuration “O” between two adjacent posts 110 results in a change in curvature of the corresponding outwardly convex and outwardly concave regions 121, 122 of each leaflet 104. Specifically, movement of each leaflet 104 from its closed configuration “C” towards its open configuration “O” results in an initial increase in the curvature of the corresponding outwardly convex region 121 and the corresponding outwardly concave region 122 of each leaflet 104 followed by a decrease in the curvature of the outwardly convex region 121 of each leaflet 104 and a further increase in the curvature of the outwardly concave region 122 of each leaflet 104 such that the outwardly convex region 121 of the leaflet 104 appears to “grow” at the expense of the outwardly concave region 122 of the leaflet 104 until each leaflet 104 reaches its corresponding open configuration “O”. More specifically, the curvature of the outwardly convex and concave portions 119, 120 of the free edge 115 of the leaflet 104 are accentuated until the point of inflection 118 along the free edge 115 crosses the straight line extending between the adjacent posts 110. Once the point of inflection 118 along the free edge 115 crosses the straight line extending between the adjacent posts 110, the curvature of the outwardly convex portion 119 of the free edge 115 reduces whilst the curvature of the outwardly concave portion 120 of the free edge 115 increases. Corresponding changes are also observed for each of the transverse cross-sections of the leaflets 104 represented by the contours 1-11.

The changes in curvature of the leaflets 104 described above result in each leaflet 104 moving continuously in a predictable manner. As a consequence of such movement, buckling or wrinkling of each leaflet 104 and the associated bending stresses may be avoided. This permits the valve 102 to be configured such that the bending stresses induced in each leaflet 104 as a consequence of such movement of each leaflet 104 do not exceed a threshold bending stress so that damage such as tearing of each leaflet 104 is thereby avoided.

As may be appreciated from FIGS. 2 and 3, when viewed from an outflow side of the heart valve 102 looking towards the free edges 115 of the leaflets 104, the outwardly convex region 121 of each leaflet 104 precedes the corresponding outwardly concave region 122 of said leaflet 104 in a counter-clockwise direction defined around the axis 111. More specifically, when viewed from an outflow side of the heart valve 102 looking towards the free edges 115 of the leaflets 104, the outwardly convex portion 119 of any one of the transverse cross-sections of each leaflet 104 precedes the corresponding outwardly concave portion 120 of said one of the transverse cross-sections of said leaflet 104 in a counter-clockwise direction defined around the axis 111 e.g. when viewed from an outflow side of the heart valve 102 looking towards the free edges 115 of the leaflets 104, the outwardly convex portion 119 of any one of the transverse cross-sections of each leaflet 104 represented by any one of the contours 1-11 precedes the corresponding outwardly concave portion 120 of said one of the transverse cross-sections of said leaflet 104 in a counter-clockwise direction defined around the axis 111. When compared to leaflets having a cusp-like shape like the leaflets of a natural heart valve, such leaflets 104 may open more readily in response to a helical blood flow incident on the inflow side of the heart valve 102 wherein blood flows along a forward direction whilst rotating in a counter-clockwise direction when viewed from the outflow side of the heart valve 102 looking towards the free edges 115 of the leaflets 104. When compared to leaflets having a cusp-like shape like the leaflets of a natural heart valve, such leaflets 104 may also close more readily in response to a helical blood flow incident on the outflow side of the heart valve 102 wherein blood flows along a backward direction which is opposite to the forward direction whilst continuing to rotate in the counter-clockwise direction when viewed from the outflow side of the heart valve 102 looking towards the free edges 115 of the leaflets 104. It has also been found that when compared to leaflets having a cusp-like shape like the leaflets of a natural heart valve, such leaflets 104 may open more readily in response to a helical blood flow incident on the inflow side of the heart valve 102 wherein blood flows along a forward direction whilst rotating in a clockwise direction when viewed from the outflow side of the heart valve 102 looking towards the free edges 115 of the leaflets 104. Similarly, it has also been found that when compared to leaflets having a cusp-like shape like the leaflets of a natural heart valve, such leaflets 104 may also close more readily in response to a helical blood flow incident on the outflow side of the heart valve 102 wherein blood flows along a backward direction which is opposite to the forward direction whilst continuing to rotate in the clockwise direction when viewed from the outflow side of the heart valve 102 looking towards the free edges 115 of the leaflets 104. As such, it should be understood that the shape of the leaflets 104 may work synergistically with the natural helical flow of blood to improve the efficiency of operation of the heart valve 102 when the heart valve 102 is installed and/or implanted in the human or animal body.

The design of the valve 102 also permits use of stiffer more durable, biocompatible materials for the leaflets 104 so as to provide a reduced restriction to blood flow in the open configuration “O” compared with conventional synthetic polymer heart valves made from such stiffer materials whilst also reducing susceptibility to tearing of the leaflets 104. This improved immunity to damage is attributable not only to the increased stiffness of the leaflets 104, but also to the shape of the leaflets 104. More specifically, the stiffness and the arrangement of each leaflet 104 means that, although the curvature of each leaflet 104 may change in response to changes in pressure differential across the leaflets 104, the outwardly convex and concave regions 121, 122 of the leaflets 104 generally persist for different pressure differentials across the leaflets 104. As a consequence of such movement, stresses in the leaflets 104 are distributed across the widths of the leaflets 104 and the commissural regions of the leaflets 104 in the vicinity of the frame 106 do not have to bend as much as the commissural regions of leaflets of conventional synthetic heart valves for a given outflow orifice size.

The stiffness and the arrangement of each leaflet 104 also means that each leaflet 104 has a predetermined shape for a given pressure differential across the leaflet 104. The predetermined shape of each leaflet 104 for a given pressure differential across the leaflet 104 is selected so as to prevent arbitrary buckling or wrinkling of each leaflet 104, thus avoiding excessive bending stresses in each leaflet 104. In particular, each leaflet 104 is formed so as to have a predetermined shape throughout movement of the leaflet 104 between the open and closed configurations.

The design of the synthetic heart valve 102 represents a significant departure from the design of a natural heart valve which has evolved naturally over millions of years and which works well for a life-time, but which relies for this on the physical and biological characteristics of the complex leaflet structure, composed of collagen, elastin and glycoprotein matrix, as well as the living nature of the tissue that is able to repair and replace itself. Furthermore, the principles of design for the synthetic heart valve 102 are contrary to the principles of design employed for conventional man-made heart valve designs which dictate that such conventional man-made heart valve designs should mimic natural heart valve designs. In particular, the design of synthetic heart valve 102 differs appreciably from conventional man-made heart valve designs that mimic natural heart valve designs at least by virtue of the generally straight lines of attachment along which each leaflet 104 is connected to the adjacent posts 110 of the frame 106 and by virtue of the specific characteristic shape of each of the leaflets 104.

However, for certain leaflet materials and shapes and, under certain conditions, when the leaflets 104 of the artificial heart valve 102 are in the closed configuration and are exposed to back pressure, the free edges 115 of the leaflets 104 may be susceptible to displacement in a direction of the back pressure. For example, referring to FIG. 7A, there is shown a schematic illustration of the artificial heart valve 102 when the leaflets 104 are in the open configuration. FIG. 7B is a schematic illustration of the artificial heart valve 102 when the leaflets 104 are in the closed configuration but before the leaflets 104 are exposed to back pressure and FIG. 7C is a schematic illustration of the artificial heart valve 102 when the leaflets 104 are in the closed configuration and the leaflets 104 are exposed to back pressure. As shown in FIG. 7C, when the leaflets 104 are in the closed configuration and the leaflets 104 are exposed to back pressure, a middle portion of each of the free edges 115 of the leaflets 104 may be displaced in the direction of the back pressure, potentially increasing stress in, and/or causing buckling of, a central region 160 of each leaflet 104. The increased stress in, and/or buckling of, the central region 160 of each leaflet 104 may reduce the robustness and/or lifetime of the heart valve 102.

### SUMMARY

According to an aspect of the present disclosure there is provided an artificial heart valve comprising:
a frame;
a plurality of flexible leaflets attached to the frame; and
a plurality of support members, each support member being attached to a corresponding leaflet,
wherein the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein the support members are movable with the leaflets relative to the frame, and
wherein, when the leaflets are in the closed configuration and exposed to a back pressure having at least a component in the backward direction, a force is exerted on the leaflets in the backward direction, and the support members at least partially prevent or resist deformation of the leaflets.

The support members may prevent or resist deformation of the leaflets in the form of displacement of the leaflets relative to the frame in the backward direction.

The support members may prevent or resist deformation of the leaflets by increasing the rigidity of the leaflet in locations prone to buckling. Additionally, in some cases, the support members may at least partially transfer the force from the leaflets to the frame to prevent or resist deformation of the leaflets.

Such an artificial heart valve may be less susceptible to deformation or displacement of the leaflets of the artificial heart valve in the backward direction when the leaflets are in the closed configuration and the leaflets are exposed to back pressure. Consequently, when the leaflets are in the closed configuration and the leaflets are exposed to back pressure, each leaflet may experience less stress and/or reduced buckling compared with the degree of stress and/or buckling experienced by each leaflet of a known artificial heart valve of the kind described above. Thus, such an artificial heart valve may be more robust and/or have a greater lifetime than the known artificial heart valve of the kind described above.

The frame may comprise an annular base. The frame may comprise two or more posts.

Each leaflet may comprise a base portion attached to the base of the frame. Side portions of each leaflet may be attached to corresponding adjacent posts of the frame. Each leaflet may have a free edge which extends between the corresponding adjacent posts of the frame, opposite the base of the frame. Each leaflet may have a co-aptation region which extends from the free edge of the leaflet to a boundary of the co-aptation region.

Optionally, each leaflet comprises an outwardly convex region, an outwardly concave region, and a demarcation line, wherein the outwardly convex and the outwardly concave regions meet along the demarcation line. Each leaflet may comprise a primary vertex at the point where the demarcation line intersects a base edge of the leaflet.

The outwardly convex region may comprise a region of an outer surface of a corresponding cone such as a right cone, an oblique cone or an elliptic cone. The outwardly concave region may comprise a region of an inner surface of a corresponding cone such as a right cone, an oblique cone or an elliptic cone.

Optionally, the artificial heart valve is configured such that movement of the leaflets results in a change in a curvature of the outwardly convex and outwardly concave regions of each leaflet.

Optionally, the artificial heart valve is configured such that movement of the leaflets from the closed configuration to the open configuration results in an initial increase in the curvature of the outwardly convex region and the outwardly concave region of each leaflet followed by a decrease in the curvature of the outwardly convex region and a further increase in the curvature of the outwardly concave region of each leaflet.

Optionally, in the open configuration the leaflets define an outflow orifice.

Optionally, the artificial heart valve is configured such that movement of any one of the leaflets results in a change in orientation of the corresponding demarcation line, but no change in shape of the corresponding demarcation line.

Optionally, the demarcation line of each leaflet is linear. The demarcation line may be a generator line of the cone of the outwardly concave region. The demarcation line may be a generator line of the cone of the outwardly convex region. The demarcation line may be a generator line of the cone of the outwardly concave region and a generator line of the cone of the outwardly convex region, e.g. the demarcation line may be a coincidental generator line of the cone of the outwardly concave region and the cone of the outwardly convex region. Alternatively, the demarcation line may be a generator line of the outwardly concave region only. This may permit the length of the free edge of the leaflets, and in particular the length of the free edge of the outwardly convex portion of the leaflets to be extended, thus permitting a larger outflow orifice when the leaflets are in the open configuration and encouraging helical flow through the valve.

Optionally, the demarcation line of each leaflet comprises a plurality of linear portions.

Optionally, the demarcation line may comprise first and second linear portions. The first linear portion may extend from the base portion of the leaflet to the boundary of the co-aptation region of the leaflet. The second linear portion may extend from the boundary of the co-aptation region of the leaflet to the free edge of the leaflet, e.g. the second linear portion may extend across the co-aptation region.

Optionally, the demarcation line of each leaflet is piecewise linear.

Optionally, each support member is attached to the corresponding leaflet at the demarcation line. Optionally, each support member extends at least part way along the demarcation line of the corresponding leaflet. The inventors have realised that, as each leaflet moves between its open and closed configurations, although the curvature of the each of the corresponding outwardly convex and outwardly concave regions changes and an orientation of the corresponding demarcation line varies, a shape of the corresponding demarcation line remains constant. More specifically, as each leaflet moves between its open and closed configurations, although the curvature of each of the corresponding outwardly convex and outwardly concave regions change and the corresponding demarcation line tilts relative to the adjacent post, the corresponding demarcation line does not change shape. Consequently, the inventors have realised that when each support member is aligned with a corresponding demarcation line, each support member may tilt relative to the adjacent post to accommodate movement of, including changes in curvature in, the corresponding leaflet without unduly restricting the movement of the leaflet between its open and closed configurations.

Each support member may extend along the first linear portion of the demarcation line of the corresponding leaflet.

The artificial heart valve may further comprise a plurality of supplementary support members, at least one supplementary support member being attached to a corresponding leaflet.

The plurality of supplementary support members may be configured as per the plurality of the support members except for the location of the supplementary support members.

The supplementary support members may be movable with the leaflets relative to the frame.

When the leaflets are in the closed configuration and exposed to a back pressure having at least a component in the backward direction the supplementary support members may at least partially prevent or resist deformation of the leaflets. The supplementary support members may prevent or resist deformation of the leaflets by increasing the rigidity of the leaflet in locations prone to buckling.

Optionally, at least one supplementary support member is attached to the outwardly convex region of the corresponding leaflet. At least one supplementary support member may extend along a generator line of the cone of the outwardly convex region of the corresponding leaflet.

Optionally, at least one supplementary support member is attached to the outwardly concave region of the corresponding leaflet. At least one supplementary support member may extend along a generator line of the cone of the outwardly concave region of the corresponding leaflet.

Optionally, at least one supplementary support member extends along a generator line of the cone of the outwardly convex region or the cone of the outwardly concave region other than the generator line of the demarcation line. Preferably the least one supplementary support member extends along a generator line of the cone of the outwardly convex region or the cone of the outwardly concave region proximal the demarcation line in order to support the area of transition between the outwardly convex and outwardly concave regions which may be most prone to buckling. The at least one supplementary support member extends along a generator line of the cone of the outwardly convex region or the cone of the outwardly concave region that intersects the demarcation line. The at least one supplementary support member extends along a generator line of the cone of the outwardly convex region or the cone of the outwardly concave region that intersects the primary vertex of the corresponding leaflet. Preferably, the artificial heart valve may comprise the plurality of supplementary support members when the demarcation line is a generator line of the outwardly concave region only. In which case, at least one supplementary support member may extend along a generator line of the cone of the outwardly convex region. This may ensure sufficient resistance to deformation of the leaflet whilst permitting the length of the free edge of the outwardly convex portion of the leaflet to be extended, thus permitting a larger outflow orifice when the leaflets are in the open configuration and encouraging helical flow through the valve. Optionally, each support member and/or supplementary support member is attached to an inner surface or an outer surface of the corresponding leaflet, preferably the outer surface of the corresponding leaflet to minimise the impact on blood flow through the outflow orifice when the leaflets are in the open configuration.

Optionally, each support member and/or supplementary support member is at least partially embedded within the corresponding leaflet.

Optionally, each support member and/or supplementary support member is at least partially inserted within the corresponding leaflet.

Optionally, each leaflet comprises a socket or a pocket for receiving the corresponding support member and/or supplementary support member.

Optionally, each support member and/or supplementary support member is formed on, for example deposited on, or applied to, an inner surface and/or an outer surface of the corresponding leaflet.

Optionally, each support member and/or supplementary support member is elongated.

Optionally, each support member and/or supplementary support member is linear.

Optionally, each support member and/or supplementary support member comprises a plurality of linear portions.

Optionally, each support member and/or supplementary support member is piecewise linear.

Optionally, each support member and/or supplementary support member is resistant to compression along a length thereof.

Optionally, each support member and/or supplementary support member is more rigid than the corresponding leaflet.

Optionally, each support member and/or supplementary support member comprises a base end and a free end, and wherein the free end of each support member and/or supplementary support member is movable with the corresponding leaflet over a greater range of movement than the corresponding base end.

Optionally, each leaflet comprises a socket or a pocket for receiving the free end of the corresponding support member and/or supplementary support member Optionally, the free end of each support member and/or supplementary support member coincides with the free edge of the corresponding leaflet. Alternatively, the free end of each support member and/or supplementary support member may coincide with the boundary of the co-aptation region of the corresponding leaflet. This may permit the co-aptation regions of the leaflets to engage one another so as to reduce or prevent blood flow between the leaflets in the backward direction.

Optionally, the free end of each support member and/or supplementary support member is located adjacent to the free edge of the corresponding leaflet, but offset from the free edge of the corresponding leaflet in the backward direction. The offset between the free end of the support member and/or supplementary support member and the free edge of the corresponding leaflet may be between 1 mm and 5 mm. The offset between the free end of the support member and/or supplementary support member and the free edge of the corresponding leaflet may be approximately 2 mm. This may at least partially permit the co-aptation regions of the leaflets to engage one another so as to reduce or prevent blood flow between the leaflets in the backward direction.

Optionally, the free ends of the support members support one another when the leaflets are in the closed configuration but do not support one another when the leaflets are in the open configuration.

Optionally, the free ends of the support members engage one another when the leaflets are in the closed configuration but are disengaged when the leaflets are in the open configuration.

Optionally, the free ends of the support members are complementary in shape to one another. When the free ends of the support members are complementary in shape to one another, the free ends of the support members may fit together better or more closely so as to increase a degree of contact between the free ends of the support members and thereby increase a degree of support between the free ends of the support members.

Optionally, the base end of each support member and/or supplementary support member is disposed towards the frame. The base end of each support member and/or supplementary support member may be proximal the primary vertex of the leaflet.

Optionally, the base end of each support member and/or supplementary support member is only attached to the frame indirectly by virtue of the attachment of the support member to the corresponding leaflet and attachment of the corresponding leaflet to the frame.

Optionally, the base ends of the support members engage, e.g. abut, corresponding engagement regions of the frame when the leaflets are in the closed configuration and the leaflets are exposed to the back pressure, wherein the base ends of the support members and the corresponding engagement regions of the frame are complementary in shape. For example, the base ends of the support members may have a convex or rounded shape and the corresponding engagement regions of the frame may have a concave shape.

Optionally, the base end of each support member is pivotally attached to the frame directly by a corresponding pivot joint.

Optionally, the base end of each support member and the frame may define a pivot joint therebetween

Optionally, each pivot joint comprises a ball and socket joint or a hinge joint.

Optionally, each pivot joint is formed integrally with the frame, for example using an additive manufacturing technique such as 3D printing.

Optionally, the base end of each support member is supported by the frame when the leaflets are in the closed configuration but is not supported by the frame when the leaflets are in the open configuration.

Optionally, the base end of each support member engages the frame when the leaflets are in the closed configuration but is disengaged from the frame when the leaflets are in the open configuration.

Optionally, the support members form a support structure with the frame for supporting the leaflets when the leaflets are in the closed configuration.

Alternatively, the base end of each support member may be spaced from the frame in order to maximise movement of the support member and leaflet relative to the frame. There may be a gap between the base end of each support member and the frame. The gap between the base end of each support member and the frame may be between 1 mm and 5 mm. The gap between the base end of each support member and the frame may be approximately 2 mm.

Optionally, the base end of each supplementary support member is spaced from the frame in order to maximise movement of the supplementary support member and leaflet relative to the frame. There may be a gap between the base end of each supplementary support member and the frame. The gap between the base end of each supplementary support member and the frame may be the same as or larger than the gap between the base end of each support member and the frame. The gap between the base end of each supplementary support member and the frame may be between 1 mm and 5 mm. The gap between the base end of each supplementary support member and the frame may be approximately 2 mm.

Optionally, each support member and/or supplementary support member comprises a corresponding base portion extending adjacent to the corresponding base end and a corresponding main body portion extending from the corresponding base portion to the corresponding free end.

Optionally, the base portion of each support member and/or supplementary support member is less rigid than the corresponding main body portion. Such support members and/or supplementary support member may result in less restriction or greater flexibility in the movement of the region of each leaflet adjacent to the base portion of the corresponding support member.

Optionally, the base portion of each support member and/or supplementary support member is tapered from the corresponding main body portion to the corresponding base end.

Optionally, the base portion of each support member and/or supplementary support member has a smaller transverse cross-section than the corresponding main body portion.

Optionally, the base portion of each support member and/or supplementary support member is thinner and/or narrower than the corresponding main body portion.

Optionally, the base portion of each support member and/or supplementary support member comprises, or is formed from, a less rigid material than a material of the corresponding main body portion.

Optionally, the base portion and the main body portion of each support member and/or supplementary support member have different degrees of isotropy.

Optionally, each support member and/or supplementary support member comprises, or is formed from, titanium, carbon fibre, graphene, or a polymeric material such as polyurethane.

Optionally, each support member and/or supplementary support member comprises a core and a coating. The coating may allow for neutral inclusion of the support member and/or supplementary support member such that the support member and/or supplementary support member may not impact stress distributions, e.g. cause stress concentrations, in the leaflet. The core may be cylindrical. The core may have a thickness, e.g. diameter, less than 1 mm. The core may have a thickness, e.g. diameter, of between 0.1 mm and 0.5 mm. The core may have a thickness, e.g. diameter, of approximately 0.3 mm. The core may comprise or be formed of a first material. The coating may have a thickness equal to or larger than the thickness of the core. The coating may have a thickness up to 1.5 times larger than a radius of the core. A diameter of the support member and/or supplementary support member may be up to 2.5 times the diameter of the core. The coating may have a thickness between 0.3 mm and 0.7 mm. The coating may have a thickness of approximately 0.5 mm. The coating may be formed of a second material. The second material may have a lower stiffness than the first material. The first material may be titanium, carbon fibre, graphene, etc. The second material may be a polymeric material such as polyurethane.

Optionally, each leaflet is flat or generally flat when formed.

Optionally, each leaflet is formed from a corresponding flat sheet of flexible material, for example by cutting the corresponding flat sheet of flexible material. The flexible material may have a lower stiffness than the first material and the second material of the support member and/or supplementary support member.

Optionally, each leaflet comprises, or is formed from, a flexible synthetic material.

Optionally, each leaflet comprises, or is formed from, a flexible polymer material.

Optionally, each leaflet comprises, or is formed from, polyurethane.

Optionally, each leaflet comprises, or is formed from, a flexible composite material which includes a flexible matrix material and one or more flexible reinforcing elements embedded within the flexible matrix material, wherein the one or more flexible reinforcing elements are distinct from the corresponding supporting member.

Optionally, each leaflet comprises, or is formed from, lower modulus polyurethane and each support member comprises, or is formed from, higher modulus polyurethane.

Optionally, the frame comprises, or is formed from, at least one of a metal, titanium, stainless steel, a polymer or polyether ether ketone (PEEK).

Optionally, the base portion of each leaflet comprises one or more holes for attaching the leaflet to the frame.

Optionally, each of the one or more holes of the base portion of each leaflet is configured to receive a corresponding pin, a corresponding peg, or a corresponding protrusion of the frame.

Optionally, each of the one or more holes of the base portion of each leaflet is configured to receive a suture, a thread, or a string for attaching the base portion of the leaflet to the frame.

Optionally, each of the first and second side portions of each leaflet comprises one or more holes for attaching the leaflet to the frame.

Optionally, each of the one or more holes of the first and second side portions of each leaflet is configured to receive a corresponding pin, a corresponding peg, or a corresponding protrusion of the frame.

Optionally, each of the one or more holes of the first and second side portions of each leaflet is configured to receive a suture, a thread, or a string for attaching the first and second side portions of the leaflet to the frame.

Optionally, when in the open configuration, the leaflets permit linear blood flow therebetween along the forward direction, and when in the closed configuration, the leaflets prevent or restrict linear blood flow therebetween along the backward direction.

Optionally, when in the open configuration, the leaflets permit helical blood flow therebetween along the forward direction, and when in the closed configuration, the leaflets prevent or restrict helical blood flow therebetween along the backward direction.

Optionally, when viewed from an outflow side of the heart valve looking towards the free edges of the leaflets, the outwardly convex and outwardly concave regions of each leaflet are arranged in an order which is commensurate with known physiological blood flow conditions. For example, when viewed from an outflow side of the heart valve looking towards the free edges of the leaflets, the outwardly convex region of each leaflet may precede the corresponding outwardly concave portion of said leaflet in a counter-clockwise direction defined around the axis. When compared to leaflets having a cusp-like shape like the leaflets of a natural heart valve, such leaflets may open more readily in response to a helical blood flow incident on the inflow side of the heart valve wherein blood flows along a forward direction whilst rotating in a counter-clockwise direction when viewed from the outflow side of the heart valve looking towards the free edges of the leaflets. When compared to leaflets having a cusp-like shape like the leaflets of a natural heart valve, such leaflets may also close more readily in response to a helical blood flow incident on the outflow side of the heart valve wherein blood flows along a backward direction which is opposite to the forward direction whilst continuing to rotate in the counter-clockwise direction when viewed from the outflow side of the heart valve looking towards the free edges of the leaflets. It has also been found that when compared to leaflets having a cusp-like shape like the leaflets of a natural heart valve, such leaflets may open more readily in response to a helical blood flow incident on the inflow side of the heart valve wherein blood flows along a forward direction whilst rotating in a clockwise direction when viewed from the outflow side of the heart valve looking towards the free edges of the leaflets. Similarly, it has also been found that when compared to leaflets having a cusp-like shape like the leaflets of a natural heart valve, such leaflets may also close more readily in response to a helical blood flow incident on the outflow side of the heart valve wherein blood flows along a backward direction which is opposite to the forward direction whilst continuing to rotate in the clockwise direction when viewed from the outflow side of the heart valve looking towards the free edges of the leaflets. As such, it should be understood that the shape of the leaflets may work synergistically with the natural helical flow of blood to improve the efficiency of operation of the heart valve when the heart valve is installed and/or implanted in the human or animal body.

Optionally, when viewed from an outflow side of the heart valve looking towards the free edges of the leaflets, the outwardly concave region of each leaflet precedes the corresponding outwardly convex region of said leaflet in a counter-clockwise direction defined around the axis. When compared to leaflets having a cusp-like shape like the leaflets of a natural heart valve, such leaflets may open more readily in response to a helical blood flow incident on the inflow side of the heart valve wherein blood flows along a forward direction whilst rotating in a clockwise direction when viewed from the outflow side of the heart valve looking towards the free edges of the leaflets. When compared to leaflets having a cusp-like shape like the leaflets of a natural heart valve, such leaflets may also close more readily in response to a helical blood flow incident on the outflow side of the heart valve wherein blood flows along a backward direction which is opposite to the forward direction whilst continuing to rotate in the clockwise direction when viewed from the outflow side of the heart valve looking towards the free edges of the leaflets. It has also been found that when compared to leaflets having a cusp-like shape like the leaflets of a natural heart valve, such leaflets may open more readily in response to a helical blood flow incident on the inflow side of the heart valve wherein blood flows along a forward direction whilst rotating in a counter-clockwise direction when viewed from the outflow side of the heart valve looking towards the free edges of the leaflets. Similarly, it has also been found that when compared to leaflets having a cusp-like shape like the leaflets of a natural heart valve, such leaflets may also close more readily in response to a helical blood flow incident on the outflow side of the heart valve wherein blood flows along a backward direction which is opposite to the forward direction whilst continuing to rotate in the counter-clockwise direction when viewed from the outflow side of the heart valve looking towards the free edges of the leaflets.

Optionally, the leaflets are formed integrally on and/or around the frame so as to attach the leaflets to the frame.

Optionally, the leaflets are bonded to the frame, for example using an adhesive substance.

Optionally, the frame comprises a base defining an aperture for blood flow.

Optionally, each leaflet comprises a corresponding base portion attached to the base of the frame along a corresponding base line of attachment.

Optionally, the base portion of each leaflet extends around a corresponding portion of the aperture.

Optionally, the base line of attachment of each leaflet extends around a corresponding portion of the aperture.

Optionally, the base portion of each leaflet is outwardly convex relative to an axis defined by the aperture.

Optionally, the base line of attachment of each leaflet is outwardly convex relative to an axis defined by the aperture.

Optionally, the base of the frame comprises one or more holes extending therethrough, wherein the base portion of each leaflet extends through one or more of the holes so as to attach the leaflets to the frame.

Optionally, the base portion of each leaflet comprises one or more holes extending therethrough and the base of the frame comprises one or more pins, projections or protrusions extending therefrom, wherein one or more of the pins, projections or protrusions extend through one or more of the holes extending through the base portion of one or more of the leaflets.

Optionally, the frame comprises a plurality of posts extending from the base.

Optionally, each post is at least partially straight.

Optionally, each leaflet comprises first and second side portions, wherein the first and second sides portions of each leaflet are attached to corresponding first and second adjacent posts along first and second at least partially straight lines of attachment respectively.

Optionally, the first and second side portions of each leaflet are at least partially straight.

Optionally, each post comprises one or more holes extending therethrough, wherein each side portion of each leaflet extends through one or more of the holes extending through a corresponding one of the posts.

Optionally, the side portions of each leaflet comprises one or more holes extending therethrough and each post comprises one or more pins, projections or protrusions extending therefrom, wherein one or more of the pins, projections or protrusions extend through one or more of the holes extending through one or more side portions of one or more of the leaflets.

Optionally, each leaflet comprises a plurality of transverse cross-sections, wherein each transverse cross-section is taken through said leaflet in a corresponding transverse plane which is transverse to an axis defined by the aperture, and wherein each transverse plane extends through the outwardly convex region, the outwardly concave region, and the demarcation line of said leaflet so as to define a corresponding transverse cross-section which extends along a corresponding curved line and which comprises a corresponding outwardly convex portion, a corresponding outwardly concave portion, and a corresponding point of inflection respectively, wherein the corresponding outwardly convex portion is outwardly convex relative to the axis, the corresponding outwardly concave portion is outwardly concave relative to the axis, and the corresponding point of inflection is located at a corresponding junction between the corresponding outwardly convex portion and the corresponding outwardly concave portion.

Optionally, the outwardly convex portion of each transverse cross-section extends from the corresponding first post to the corresponding point of inflection, and the outwardly concave portion of each transverse cross-section extends from the corresponding second post to the corresponding point of inflection.

Optionally:
a first one of the transverse cross-sections taken through any one of the leaflets on a first one of the transverse planes comprises a first outwardly convex portion, a first outwardly concave portion, and a first point of inflection,
a second one of the transverse cross-sections taken through any one of the leaflets on a second one of the transverse planes comprises a second outwardly convex portion, a second outwardly concave portion, and a second point of inflection,
the first one of the transverse planes is located closer to the base of the frame than the second one of the transverse planes, and
the first outwardly convex portion is longer than the second outwardly convex portion and the first outwardly concave portion is shorter than the second outwardly concave portion.

Optionally, each leaflet comprises a corresponding free edge which is movable relative to the frame, wherein the corresponding free edge extends along a corresponding curved line in a transverse plane transverse to an axis defined by the aperture, and the corresponding free edge comprises a corresponding outwardly convex portion, a corresponding outwardly concave portion and a corresponding point of inflection between the corresponding outwardly convex portion and the corresponding outwardly concave portion, wherein the corresponding outwardly convex portion of the free edge extends from the corresponding first post to the corresponding point of inflection of the free edge, and the corresponding outwardly concave portion of the free edge extends from the corresponding second post to the corresponding point of inflection of the free edge.

Optionally, the point of inflection of the free edge is located half-way along, or substantially half-way along, the free edge between the corresponding first and second adjacent posts.

Optionally, the free edge of any leaflet is longer than the base portion of said leaflet.

Optionally, the free edge of any leaflet is longer than any of the transverse cross-sections of said leaflet.

Optionally, each leaflet defines a corresponding co-aptation region which extends from the corresponding free edge towards the base of the frame and which has a plurality of corresponding generally identical transverse cross-sections.

Optionally, the artificial heart valve is configured such that movement of the leaflets results in a change in a curvature of the outwardly convex portions of the outwardly convex region of any one of the leaflets and a change in a curvature of the outwardly concave portions of the outwardly concave region of said one of the leaflets.

Optionally, the artificial heart valve is configured such that movement of the leaflets from the closed configuration to the open configuration results in an initial increase in the curvature of the outwardly convex portions of the outwardly convex region of any one of the leaflets and an initial increase in the curvature of the corresponding outwardly concave portions of the outwardly concave region of said one of the leaflets followed by a decrease in the curvature of the outwardly convex portions of the outwardly convex region of said one of the leaflets and a further increase in the curvature of the corresponding outwardly concave portions of the outwardly concave region of said one of the leaflets.

Optionally, when viewed from an outflow side of the heart valve looking towards the free edges of the leaflets, the outwardly convex portion of any one of the transverse cross-sections of each leaflet precedes the corresponding outwardly concave portion of said one of the transverse cross-sections of said leaflet in a counter-clockwise direction defined around the axis.

Optionally, when viewed from an outflow side of the heart valve looking towards the free edges of the leaflets, the outwardly concave portion of any one of the transverse cross-sections of each leaflet precedes the corresponding outwardly convex portion of said one of the transverse cross-sections of said leaflet in a counter-clockwise direction defined around the axis.

Optionally, the artificial heart valve comprises:
three flexible leaflets attached to the frame; and
three support members,
wherein each support member is attached to a corresponding leaflet.

Optionally, the three support members form a tripod support structure when the three leaflets are in the closed configuration.

Optionally, the free ends of the three support members support one another when the three leaflets are in the closed configuration but do not support one another when the three leaflets are in the open configuration.

Optionally, the free ends of the three support members engage one another when the three leaflets are in the closed configuration but are disengaged when the leaflets are in the open configuration.

Optionally, the artificial heart valve further comprises three supplementary support members, wherein each support member is attached to an outwardly convex portion of a corresponding leaflet.

Optionally, the artificial heart valve comprises:
two flexible leaflets attached to the frame; and
two support members,
wherein each support member is attached to a corresponding leaflet.

Optionally, the two support members form an inverted V-shaped support structure, e.g. an A-frame support structure, when the two leaflets are in the closed configuration.

Optionally, the free ends of the two support members support one another when the two leaflets are in the closed configuration but do not support one another when the two leaflets are in the open configuration.

Optionally, the free ends of the two support members engage one another when the two leaflets are in the closed configuration but are disengaged when the two leaflets are in the open configuration.

Optionally, the artificial heart valve further comprises two supplementary support members, wherein each support member is attached to an outwardly convex portion of a corresponding leaflet.

According to an aspect of the present disclosure there is provided a leaflet arrangement for an artificial heart valve, the leaflet arrangement comprising:
a flexible leaflet configured for attachment to a frame of the artificial heart valve; and
a support member attached to the leaflet,
wherein, the leaflet arrangement is attachable to the frame together with one or more other identical leaflet arrangements, so that:
   the leaflets of the leaflet arrangements are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
   the support members of the leaflet arrangements are movable with the leaflets relative to the frame, and
   when the leaflets of the leaflet arrangements are in the closed configuration and exposed to a back pressure having at least a component in the backward direction, a force is exerted on the leaflets in the backward direction and the support members at least partially prevent or resist deformation of the leaflets.

The support members may prevent or resist displacement of the leaflets relative to the frame in the backward direction.

The support members may prevent or resist deformation of the leaflets by increasing the rigidity of the leaflet in locations prone to buckling. Additionally, in some cases, the support members may at least partially transfer the force from the leaflets to the frame to prevent or resist deformation of the leaflets.

Optionally, the leaflet arrangement is flat or generally flat.

Optionally, the leaflet arrangement further comprises a supplementary support member attached to the leaflet. The supplementary support member may be movable with the leaflet relative to the frame. When the leaflets are in the closed configuration and exposed to a back pressure having at least a component in the backward direction the supplementary support members may at least partially prevent or resist deformation of the leaflets. The supplementary support members may prevent or resist deformation of the leaflets by increasing the rigidity of the leaflet in locations prone to buckling.

Optionally, the support member and/or supplementary support member is attached to an inner surface or an outer surface of the leaflet.

Optionally, the support member and/or supplementary support member is at least partially embedded within the leaflet.

Optionally, the support member and/or supplementary support member is at least partially inserted within the leaflet.

Optionally, the leaflet comprises a socket or a pocket for receiving a free end of the support member and/or supplementary support member, and the free end of the support member and/or supplementary support member is inserted into the socket or pocket.

Optionally, the support member and/or supplementary support member is formed separately from the leaflet.

Optionally, the support member and/or supplementary support member comprises, or is formed from, a different material to the leaflet.

Optionally, the support member and/or supplementary support member is formed on, for example deposited on, or applied to, an inner surface and/or an outer surface of the leaflet.

Optionally, each support member and/or supplementary support member comprises, or is formed from, titanium, carbon fibre, graphene, or a polymeric material such as polyurethane.

Optionally, each support member and/or supplementary support member comprises a core and a coating. The core may be cylindrical. The core may have a thickness, e.g. diameter, of between 0.1 mm and 0.5 mm. The core may have a thickness, e.g. diameter, of approximately 0.3 mm. The core may comprise or be formed of a first material. The coating may have a thickness equal to or larger than the thickness of the core. The coating may have a thickness between 0.3 mm and 0.7 mm. The coating may have a thickness of approximately 0.5 mm. The coating may be formed of a second material. The second material may have a lower stiffness than the first material. The first material may be titanium, carbon fibre, graphene, etc. The second material may be a polymeric material such as polyurethane.

Optionally, each leaflet is flat or generally flat when formed.

Optionally, each leaflet is formed from a corresponding flat sheet of flexible material, for example by cutting the corresponding flat sheet of flexible material. The flexible material may have a lower stiffness than the first material and the second material of the support member and/or supplementary support member.

Optionally, each leaflet comprises, or is formed from a synthetic material.

Optionally, each leaflet comprises, or is formed from, a flexible polymer material.

Optionally, each leaflet comprises, or is formed from, polyurethane.

Optionally, each leaflet comprises, or is formed from, a flexible composite material which includes a flexible matrix material and one or more flexible reinforcing elements embedded within the flexible matrix material, wherein the one or more flexible reinforcing elements are distinct from the corresponding supporting member.

Optionally, each leaflet comprises, or is formed from, lower modulus polyurethane and each support member comprises, or is formed from, higher modulus polyurethane.

Optionally, the frame comprises, or is formed from, at least one of a metal, titanium, stainless steel, a polymer or polyether ether ketone (PEEK).

According to an aspect of the present disclosure there is provided a method of manufacturing an artificial heart valve, the method comprising:
attaching a corresponding support member to each flexible leaflet of a plurality of flexible leaflets, and
attaching each leaflet to a frame,
wherein the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein the support members are movable with the leaflets relative to the frame, and
wherein, when the leaflets are in the closed configuration and exposed to a back pressure having at least a component in the backward direction, a force is exerted on the leaflets in the backward direction and the support members at least partially prevent or resist deformation of the leaflets.

The support members may prevent or resist displacement of the leaflets relative to the frame in the backward direction.

The support members may prevent or resist deformation of the leaflets by increasing the rigidity of the leaflet in locations prone to buckling. Additionally, in some cases, the support members may at least partially transfer the force from the leaflets to the frame to prevent or resist deformation of the leaflets.

Optionally, the method comprises forming each leaflet separately from the frame and then attaching each leaflet to the frame.

Optionally, the method comprises forming each leaflet as a flat sheet.

Optionally, the method comprises forming each leaflet from a flat sheet of flexible material, for example by cutting the corresponding flat sheet of flexible material.

Optionally, the method comprises forming each leaflet on the frame, for example by moulding.

Optionally, the method comprises attaching a corresponding supplementary support member to each flexible leaflet of a plurality of flexible leaflets. The supplementary support members may be movable with the leaflets relative to the frame. When the leaflets are in the closed configuration and exposed to a back pressure having at least a component in the backward direction the supplementary support members may at least partially prevent or resist deformation of the leaflets. The supplementary support members may prevent or resist deformation of the leaflets by increasing the rigidity of the leaflet in locations prone to buckling.

Optionally, the method comprises attaching each support member and/or supplementary support member to an inner surface or an outer surface of the corresponding leaflet.

Optionally, the method comprises at least partially embedding each support member and/or supplementary support member within the corresponding leaflet.

Optionally, the method comprises at least partially inserting each support member and/or supplementary support member within the corresponding leaflet.

Optionally, each leaflet comprises a socket or a pocket for receiving the free end of the corresponding support member and/or supplementary support member and the method comprises inserting the free end of the corresponding support member and/or supplementary support member into the socket or pocket.

Optionally, the method comprises forming each support member and/or supplementary support member separately from the corresponding leaflet.

Optionally, the method comprises forming each support member and/or supplementary support member on, for example depositing each support member and/or supplementary support member on, or applying each support member and/or supplementary support member to, an inner surface and/or an outer surface of the corresponding leaflet.

According to an aspect of the present disclosure there is provided a method of manufacturing an artificial heart valve, the method comprising:
forming each leaflet of a plurality of flexible leaflets separately from a frame; and then
attaching each leaflet to the frame so that the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein forming each leaflet separately from the frame comprises forming each leaflet as a flat sheet or forming each leaflet from a flat sheet of flexible material.

Forming each leaflet as a flat sheet or from a flat sheet of material allows for greater control and consistency in manufacturing.

Optionally, forming each leaflet comprises cutting a corresponding flat sheet of flexible material.

Optionally, forming each leaflet comprises forming each leaflet from a synthetic material.

Optionally, forming each leaflet comprises forming each leaflet from a flexible polymer material.

Optionally, forming each leaflet comprises forming each leaflet from polyurethane.

Optionally, forming each leaflet comprises forming each leaflet from a flexible composite material which includes a flexible matrix material and one or more flexible reinforcing elements embedded within the flexible matrix material.

Optionally, the frame comprises, or is formed from, at least one of a metal, titanium, stainless steel, a polymer or polyether ether ketone (PEEK).

Optionally, when attached to the frame, each leaflet comprises an outwardly convex region, an outwardly concave region, and a demarcation line, wherein the outwardly convex and the outwardly concave regions meet along the demarcation line. The outwardly convex region may comprise a region of an outer surface of a corresponding cone such as a right cone, an oblique cone or an elliptic cone. The outwardly concave region may comprise a region of an inner surface of a corresponding cone such as a right cone, an oblique cone or an elliptic cone.

It is not generally possible to form a leaflet separately from a frame as a flat sheet or from a flat sheet of flexible material and to then attach the leaflet to the frame so as to form a “cusp-shaped” leaflet. However, when each leaflet is configured so that, when attached to the frame, each leaflet comprises an outwardly convex region, an outwardly concave region, and a demarcation line, wherein the outwardly convex and the outwardly concave regions meet along the demarcation line, the inventors have discovered that each leaflet may be formed separately from the frame as a flat sheet or may be formed from a flat sheet of flexible material before being attached to the frame. This may simplify the manufacture of the artificial heart valve.

According to an aspect of the present disclosure there is provided an artificial heart valve comprising:
a frame;
a plurality of flexible leaflets attached to the frame; and
a plurality of support members, each support member being attached to a corresponding leaflet,
wherein the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein the support members are movable with the leaflets relative to the frame, and
wherein, when the leaflets are in the closed configuration and exposed to a back pressure having at least a component in the backward direction, a force is exerted on the leaflets in the backward direction, and the support members at least partially transfer the force from the leaflets to the frame to thereby prevent or resist displacement of the leaflets relative to the frame in the backward direction.

According to an aspect of the present disclosure there is provided a leaflet arrangement for an artificial heart valve, the leaflet arrangement comprising:
a flexible leaflet configured for attachment to a frame of the artificial heart valve; and
a support member attached to the leaflet,
wherein, the leaflet arrangement is attachable to the frame together with one or more other identical leaflet arrangements, so that:
   the leaflets of the leaflet arrangements are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
   the support members of the leaflet arrangements are movable with the leaflets relative to the frame, and
   when the leaflets of the leaflet arrangements are in the closed configuration and exposed to a back pressure having at least a component in the backward direction, a force is exerted on the leaflets in the backward direction and the support members at least partially transfer the force from the leaflets to the frame to thereby prevent or resist displacement of the leaflets relative to the frame in the backward direction.

According to an aspect of the present disclosure there is provided a method of manufacturing an artificial heart valve, the method comprising:
attaching a corresponding support member to each flexible leaflet of a plurality of flexible leaflets, and
attaching each leaflet to a frame,
wherein the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein the support members are movable with the leaflets relative to the frame, and
wherein, when the leaflets are in the closed configuration and exposed to a back pressure having at least a component in the backward direction, a force is exerted on the leaflets in the backward direction and the support members at least partially transfer the force from the leaflets to the frame to thereby prevent or resist displacement of the leaflets relative to the frame in the backward direction.

It should be understood that any one or more of the features of any one of the foregoing aspects of the present disclosure may be combined with any one or more of the features of any of the other foregoing aspects of the present disclosure.

### BRIEF DESCRIPTION OF THE DRAWINGS

A heart valve will now be described by way of non-limiting example only with reference to the drawings of which:

FIG. 1A is a schematic perspective view of a known artificial heart valve;

FIG. 1B is a schematic perspective view of a frame of the known artificial heart valve of FIG. 1A;

FIG. 2 is a contour plot of the known artificial heart valve of FIG. 1A when viewed from an outflow side of the artificial heart valve illustrating the shape of the leaflets of the artificial heart valve when the leaflets are in a natural or default configuration;

FIG. 3 is a schematic view of the known artificial heart valve of FIG. 1A when viewed from an outflow side of the artificial heart valve illustrating the shape of the free edges of the leaflets of the artificial heart valve when the leaflets are in an open configuration, a closed configuration, and a natural or default configuration;

FIG. 4A is a schematic perspective view of an artificial heart valve;

FIG. 4B is a schematic perspective view of a frame of the artificial heart valve of FIG. 4A;

FIG. 5 is a contour plot of the artificial heart valve of FIG. 4A when viewed from an outflow side of the artificial heart valve illustrating the shape of the leaflets of the artificial heart valve when the leaflets are in a natural or default configuration;

FIG. 6 is a schematic view of the artificial heart valve of FIG. 4A when viewed from an outflow side of the artificial heart valve illustrating the shape of the free edges of the leaflets of the artificial heart valve when the leaflets are in an open configuration, a closed configuration, and a natural or default configuration;

FIG. 7A is a simplified schematic perspective view of the known artificial heart valve of FIG. 1A when the leaflets are in the open configuration;

FIG. 7B is a simplified schematic perspective view of the known artificial heart valve of FIG. 1A when the leaflets are in the closed configuration before exposure of the leaflets to back pressure;

FIG. 7C is a simplified schematic perspective view of the known artificial heart valve of FIG. 1A when the leaflets are in the closed configuration after exposure of the leaflets to back pressure;

FIG. 7D is a simplified schematic perspective view of the artificial heart valve of FIG. 4A when the leaflets are in the open configuration;

FIG. 7E is a simplified schematic perspective view of the artificial heart valve of FIG. 4A when the leaflets are in the closed configuration before exposure of the leaflets to back pressure;

FIG. 7F is a simplified schematic perspective view of the artificial heart valve of FIG. 4A when the leaflets are in the closed configuration after exposure of the leaflets to back pressure;

FIG. 8A is a perspective view of a first alternative artificial heart valve when the leaflets are in the closed configuration before exposure of the leaflets to back pressure;

FIG. 8B is a view from the outflow side of the first alternative artificial heart valve of FIG. 8A when the leaflets are in the closed configuration before exposure of the leaflets to back pressure;

FIG. 8C is a perspective view of the first alternative artificial heart valve of FIG. 8A when the leaflets are in the closed configuration after exposure of the leaflets to back pressure;

FIG. 8D is a view from the outflow side of the first alternative artificial heart valve of FIG. 8A when the leaflets are in the closed configuration after exposure of the leaflets to back pressure;

FIG. 9 is a Cartesian diagram mapping a flat leaflet arrangement of the first alternative heart valve, the leaflet arrangement comprising a flexible leaflet and a support member attached to the leaflet;

FIG. 10 is a schematic illustration of the leaflet arrangement of the first alternative heart valve;

FIG. 11A is a schematic view of a second alternative artificial heart valve when viewed from an outflow side of the artificial heart valve illustrating the shape of the free edges of the leaflets of the artificial heart valve when the leaflets are in, a closed configuration; and

FIG. 11B is a schematic view of the second alternative artificial heart valve of FIG. 11A when viewed from an outflow side of the artificial heart valve illustrating the shape of the free edges of the leaflets of the artificial heart valve when the leaflets are in an open configuration;

FIGS. 12A and 12B are Cartesian diagrams mapping an alternative flat leaflet arrangement, the leaflet arrangement comprising a flexible leaflet and a support member attached to the leaflet;

FIG. 13 is a cross-section view through a member, e.g. either a support member or a supplementary support member, of the alternative leaflet arrangement embedded in the leaflet of the alternative leaflet arrangement.

### DETAILED DESCRIPTION OF THE DRAWINGS

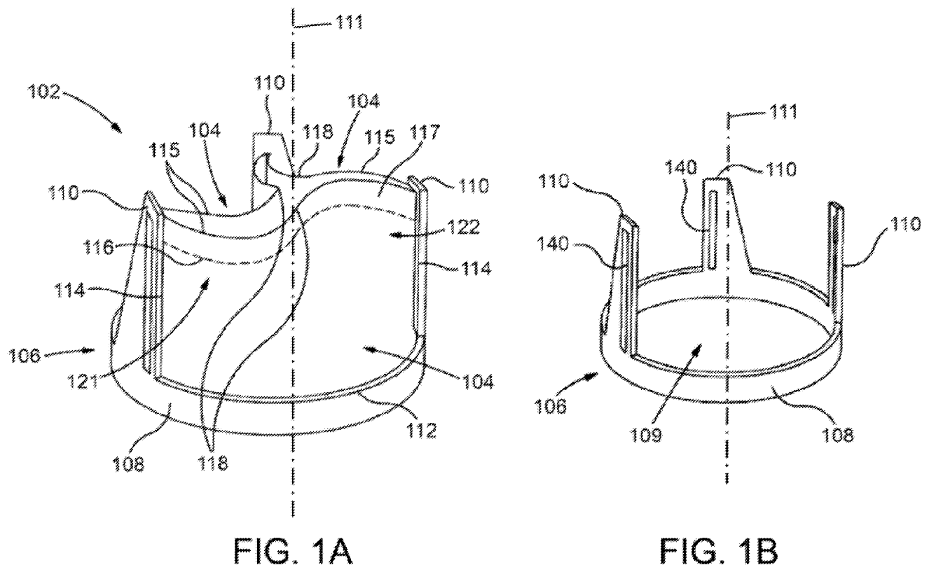

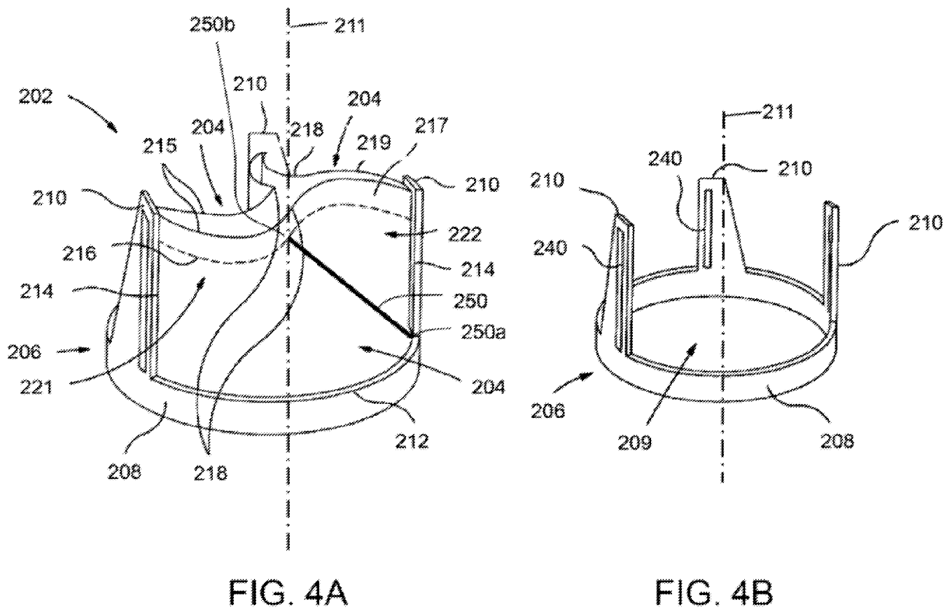

Referring to FIG. 4A, there is shown an artificial heart valve generally designated 202 for use, in particular though not exclusively in place of the aortic valve of a patient, the heart valve 202 comprising three flexible leaflets 204 attached to a frame 206. The artificial heart valve 202 of FIG. 4A includes many features which correspond closely to features of the artificial heart valve 102, wherein like features of the artificial heart valve 202 of FIG. 4A have reference numerals which are given by the reference numerals of the corresponding features of the artificial heart valve 102 of FIG. 1A incremented by “100”. The leaflets 204 may comprise, or be formed from, polyurethane. The frame 206 may comprise, or be formed from, titanium.

FIG. 4B shows the frame 206 in isolation from the leaflets 204. As may be appreciated from FIG. 4B, the frame 206 comprises a generally annular base 208 defining an aperture 209 and three posts 210 extending from the base 208. The aperture 209 defines an axis 211. The posts 210 are generally straight and extend from the base 208 in a direction which is generally parallel to the axis 211. Each post 210 defines an aperture in the form of a slit 240 for attachment of corresponding side portions of two of the leaflets 204 to the post 210.

Referring back to FIG. 4A, a base portion 212 of each leaflet 204 is outwardly convex relative to the axis 211 and extends partially around, and is attached to, the annular base 208 of the frame 206 along a corresponding base line of attachment. Side portions 214 of each leaflet 204 are attached to corresponding first and second adjacent posts 210 of the frame 206 along corresponding side lines of attachment. Each leaflet 204 has a free edge 215 which extends between the corresponding first and second adjacent posts 210 of the frame 206 opposite the base 208 of the frame 206. The free edge 215 of each leaflet 204 is free to move relative to the frame 206 in response to a difference in pressure between an inflow side of the leaflets 204 and an outflow side of the leaflets 204.

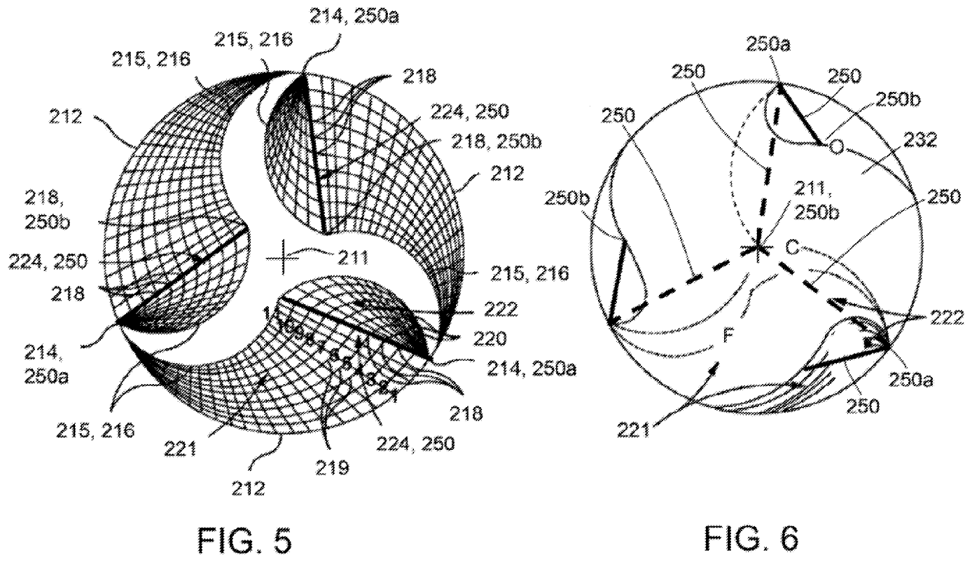

FIG. 5 shows a contour map of the leaflets 204 in their natural or as-formed configuration in which contours 1-11 represent constant height contours increasing in distance from the base 208 of the frame 206 such that contour 1 coincides with the base 208 of the frame of a leaflet 206 and contour 11 represents a boundary 216 of a co-aptation region 217 which extends from the free edge 215 of the leaflet to the boundary 216 of the co-aptation region 217. As may be appreciated from the contours shown in FIG. 5, each leaflet 204 comprises an outwardly convex region 221 and an outwardly concave region 222, wherein the corresponding outwardly convex and outwardly concave regions 221, 222 meet along a corresponding demarcation line 224. Moreover, as may be appreciated from the following description, the outwardly convex region 221 may comprise a region of an outer surface of a corresponding cone such as a right cone, an oblique cone or an elliptic cone. Similarly, as may be appreciated from the following description, the outwardly concave region 222 may comprise a region of an inner surface of a corresponding cone such as a right cone, an oblique cone or an elliptic cone.

As shown in FIG. 5, each leaflet 204 comprises a plurality of transverse cross-sections, wherein each transverse cross-section is taken through said leaflet 204 in a corresponding transverse plane which is transverse to the axis 211 and wherein each transverse cross-section extends along a corresponding curved line in the form of one of the contours 1-11 shown in FIG. 5. Each transverse plane extends through the outwardly convex region 221, the outwardly concave region 222, and the demarcation line 224 of said leaflet 204 so as to define a corresponding outwardly convex portion 219 of the corresponding transverse cross-section, a corresponding outwardly concave portion 220 of the corresponding transverse cross-section, and a corresponding point of inflection 218 of the corresponding transverse cross-section respectively. The corresponding outwardly convex portion 219 is outwardly convex relative to the axis 211, the corresponding outwardly concave portion 220 is outwardly concave relative to the axis 211, and the corresponding point of inflection 218 is located at a corresponding junction between the corresponding outwardly convex portion 219 and the corresponding outwardly concave portion 220 so that each transverse cross-section has an “S-shape” when viewed from an outflow direction. The corresponding outwardly convex portion 219 extends from a corresponding first post 210 to the corresponding point of inflection 218, and the corresponding outwardly concave portion 220 extends from the corresponding second post 210 to the corresponding point of inflection 218. Since each leaflet 204 is attached along a generally straight side line of attachment to a post 210 of the frame 206, the contours 1-11 shown in FIG. 5 meet in the vicinity of each post 210 of the frame 206.

In addition, as may be appreciated from FIG. 5, a first one of the transverse cross-sections taken through any one of the leaflets 104 on a first one of the transverse planes comprises a first outwardly convex portion, a first outwardly concave portion, and a first point of inflection, and a second one of the transverse cross-sections taken through any one of the leaflets on a second one of the transverse planes comprises a second outwardly convex portion, a second outwardly concave portion, and a second point of inflection, wherein the first one of the transverse planes is located closer to the base 208 of the frame 206 than the second one of the transverse planes and wherein the first outwardly convex portion is longer than the second outwardly convex portion and the first outwardly concave portion is shorter than the second outwardly concave portion.

From the foregoing description, it will be appreciated that each transverse cross-section through each leaflet 204 is longer than the base portion 212 of said leaflet 204 which is attached to the base 208 of the frame 206. Similarly, the free edge 215 of each leaflet 204 is longer than the corresponding base portion 212 of said leaflet 204.

Moreover, the point of inflection 218 corresponding to the free edge 215 is located half-way along, or substantially half-way along, the free edge 215 between the corresponding first and second adjacent posts 210. Furthermore, the co-aptation region 217 of each leaflet 204 comprises a plurality of corresponding generally identical transverse cross-sections. Consequently, the demarcation line 224 comprises first and second linear portions, wherein the first linear portion extends from a primary vertex at the base 208 of the frame 206 to the point of inflection 218 of the transverse cross-section at the boundary 216 of the co-aptation region 217 and the second linear portion extends from the point of inflection 218 of the transverse cross-section at the boundary 216 of the co-aptation region 217 to the point of inflection 218 of the free edge 215.

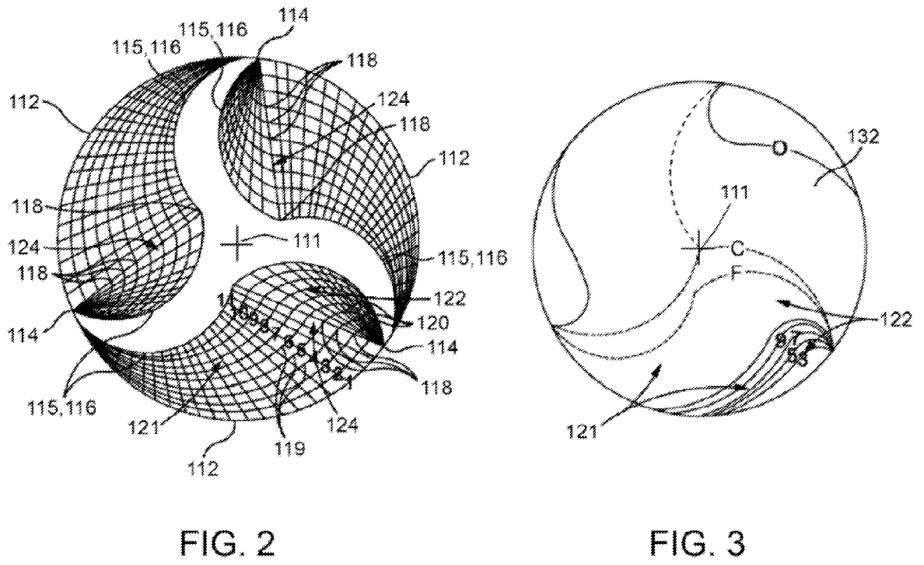

As shown in FIGS. 4A, 5 and 6 and, unlike the artificial heart valve 102 of FIGS. 1A, 2 and 3, the artificial heart valve 202 includes a plurality of linear titanium support members 250, wherein each support member 250 is attached to an outer surface of a corresponding one of the leaflets 204 relative to the axis 211 and extends part-way along the corresponding demarcation line 224 from a position adjacent to a corresponding post 210 at a base 208 of the frame 206, e.g. adjacent the primary vertex, to a mid-point on the boundary 216 of the co-aptation region 217 of the corresponding leaflet 204. Each support member 250 is resistant to compression and bending along a length thereof and is more rigid than the corresponding leaflet 204. Each support member 250 comprises a base end 250a and a free end 250b, wherein the free end 250b of each support member 250 is movable with the corresponding leaflet 204 over a greater range of movement than the corresponding base end 250a. The base end 250a of each support member 250 is disposed towards the frame 206 but is only attached to the frame 206 indirectly by virtue of the attachment of the support member 250 to the corresponding leaflet 204 and attachment of the corresponding leaflet 204 to the frame 206. The free end 250b of each support member 250 is located adjacent to the free edge 215 of the corresponding leaflet 204, but offset from the free edge 215 of the corresponding leaflet 204 in the backward direction. As shown in FIG. 4A, the free end 250b of each support member 250 is located at the boundary 216 of a co-aptation region 217.

In use, when the leaflets 204 are configured in the closed configuration so that the free edges 115 of the leaflets 104 extend along the dashed paths denoted “C” in FIG. 6 and the leaflets 204 are exposed to a back pressure acting in a backward direction on an outflow side of the leaflets 204, the radially inner surfaces of the co-aptation regions 217 of the leaflets 204 engage one another so as to reduce or prevent blood flow between the leaflets 204 in the backward direction, the free ends 250b of the support members 250 support one another, and the base end 250a of each support member 250 is supported by the frame 206 so that the support members 250 together form a support structure in the form of a tripod support structure as shown by the straight dashed lines and so that the support members 250 at least partially transfer the force exerted on the leaflets 204 to the frame 206 to thereby prevent or resist displacement of the leaflets 204 relative to the frame 206 in the backward direction. Specifically, when the leaflets 204 are configured in the closed configuration and the leaflets 204 are exposed to a back pressure acting in the backward direction, the free ends 250b of the support members 250 support one another at a position on the axis 211 at the points of inflection 218 mid-way along the boundary 216 of the co-aptation region 217 of the leaflets 204, and the base end 250a of each support member 250 is supported by the frame 206 so that the support members 250 together form a tripod support structure and the support members 250 at least partially transfer the force exerted on the leaflets 204 to the frame 206 to thereby prevent or resist displacement of a middle portion of the leaflets 204 relative to the frame 206 in the backward direction.

When the pressure exerted on the flexible leaflets 204 from an inflow side of the leaflets 204 sufficiently exceeds that exerted from the outflow side, the co-aptation regions 217 of the leaflets 204 disengage, the free ends 250b of the support members 250 no longer support one another and the leaflets 204 move radially outwardly until the free edges 215 adopt an open configuration denoted “O” in FIG. 6 to create an outflow orifice 232, the maximum size of which can be varied by the design of the S-shape that determines the length of the free edges 215 of the leaflets 204.

The free edges 215 of the leaflets 204 may adopt a natural or default configuration denoted “F” when no pressure differential exists across the leaflets 204, wherein the natural or default configuration denoted “F” is intermediate the closed configuration denoted “C” and the open configuration denoted “O”.

With reference to FIG. 6, as each leaflet 204 moves from its closed configuration “C” towards its open configuration “O” between two adjacent posts 210, the outwardly convex and concave regions 221, 222 of the leaflet 204 swing or pivot about their respective side lines of attachment at the corresponding posts 210. Moreover, movement of each leaflet 204 from its closed configuration “C” towards its open configuration “O” between two adjacent posts 210 results in a change in curvature of the corresponding outwardly convex and outwardly concave regions 221, 222 of each leaflet 204. Specifically, movement of each leaflet 204 from its closed configuration “C” towards its open configuration “O” results in an initial increase in the curvature of the corresponding outwardly convex region 221 and the corresponding outwardly concave region 222 of each leaflet 204 followed by a decrease in the curvature of the outwardly convex region 221 of each leaflet 204 and a further increase in the curvature of the outwardly concave region 222 of each leaflet 204 such that the outwardly convex region 221 of the leaflet 204 appears to “grow” at the expense of the outwardly concave region 222 of the leaflet 204 until each leaflet 204 reaches its corresponding open configuration “O”. More specifically, the curvature of the outwardly convex and concave portions 219, 220 of the free edge 215 of the leaflet 204 are accentuated until the point of inflection 218 along the free edge 215 crosses the straight line extending between the adjacent posts 210. Once the point of inflection 218 along the free edge 215 crosses the straight line extending between the adjacent posts 210, the curvature of the outwardly convex portion 119 of the free edge 215 reduces whilst the curvature of the outwardly concave portion 220 of the free edge 215 increases. Corresponding changes are also observed for each of the transverse cross-sections of the leaflets 204 represented by the contours 1-11.

Moreover, the inventors have realised that, as each leaflet 204 moves between its open and closed configurations, although the curvature of each of the corresponding outwardly convex and outwardly concave regions 221, 222 changes and the orientation of the corresponding demarcation line 224 varies, a shape of the corresponding demarcation line 224 remains constant. More specifically, as each leaflet 204 moves between its open and closed configurations, although the curvature of each of the corresponding outwardly convex and outwardly concave regions 221, 222 change and the corresponding demarcation line 224 tilts relative to the adjacent post 210, the corresponding demarcation line 224 does not change shape e.g. the portion of the corresponding demarcation line 224 extending from the base of the corresponding post 110 to the mid-point of the boundary 216 of the co-aptation region 217 remains straight.

Consequently, the inventors have realised that when each support member 250 is aligned with a corresponding demarcation line 224, each support member 250 may tilt relative to the adjacent post 210 to accommodate movement of, including changes in curvature in, the corresponding leaflet 204 without unduly restricting the movement of the leaflet 204 between its open and closed configurations.

In effect, this means that, when the pressure exerted on the flexible leaflets 204 from the inflow side of the leaflets 204 sufficiently exceeds that exerted from the outflow side, the co-aptation regions 217 of the leaflets 204 disengage and the free ends 250b of the support members 250 no longer support one another, the support members 250 no longer play any role in the operation of the heart valve 202. In view of the foregoing, the support members 250 may be considered to form a “phantom post” at a position on the axis 211 when the leaflets 204 are configured in the closed configuration and the leaflets 204 are exposed to a back pressure acting in the backward direction, wherein the phantom post only acts to support the leaflets 204 and to resist deformation and displacement of the leaflets 204 in the backward direction when the leaflets 204 are configured in the closed configuration and the leaflets 204 are exposed to the back pressure, and wherein the phantom post plays no role in the operation of the heart valve 202 when the co-aptation regions 217 of the leaflets 204 are disengaged and the free ends 250b of the support members 250 no longer support one another.

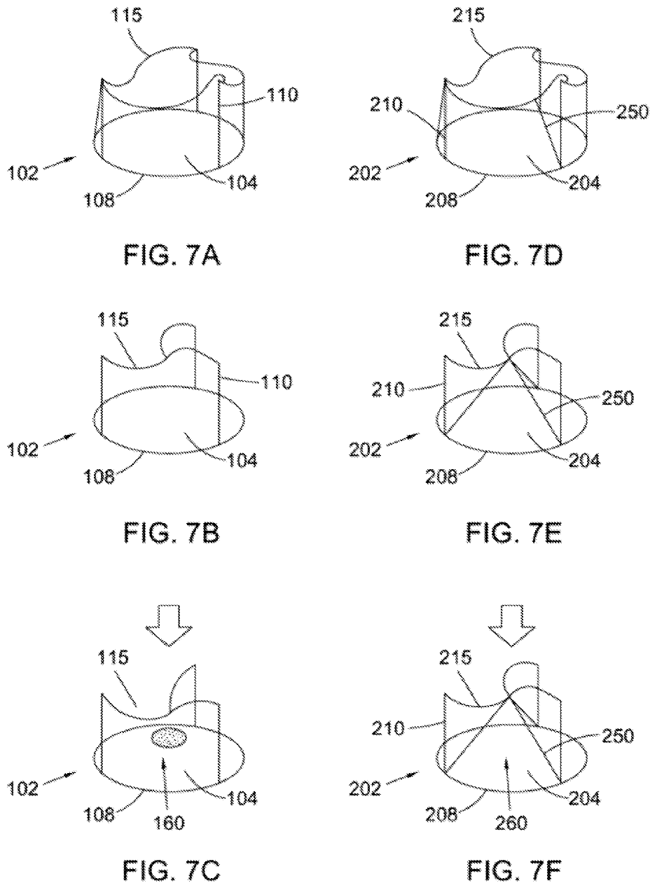

As will be understood by one of ordinary skill in the art, in addition to having the same advantages associated with the known artificial heart valve 102 described in the Background section above, the artificial heart valve 202 is also less susceptible to displacement of the middle of the leaflets 204 of the artificial heart valve 202 in the backward direction when the leaflets 204 are in the closed configuration and the leaflets 204 are exposed to back pressure. Consequently and, as may be appreciated from a comparison of FIGS. 7D, 7E and 7F with FIGS. 7A, 7B and 7C respectively, when the leaflets 204 are in the closed configuration and the leaflets 204 are exposed to back pressure, a central region 260 of each leaflet 204 may experience less stress and/or reduced buckling compared with the degree of stress and/or buckling experienced by the central region 160 of each leaflet 104 of the known artificial heart valve 102 described in the Background section above. Thus, the artificial heart valve 202 may be more robust and/or have a greater lifetime than the known artificial heart valve 102.

It should also be understood that, in practice, when the leaflets 204 are in the open configuration, the base end 250a of each support member 250 may be separated from the frame 206 by a corresponding gap which is so small that, when the leaflets 204 move towards the closed configuration and are exposed to back pressure, any displacement of the leaflets 204 in the backward direction and/or any buckling of the leaflets 204 due to movement of the base end 250a of each support member 250 across the corresponding gap towards the frame 206 is insignificant. This gap may mitigate any interference of the support member 250 with the opening and closing of the leaflets 204.

The artificial heart valve 202 may be manufactured by a variety of different methods. For example, the leaflets 204 may be formed on, or around, the frame 206 and the support members 250. For example, the leaflets 204 may be formed around the frame 206 and the support members 250 using a moulding process such as a dip moulding process whereby the frame 206 and the support members 250 are attached to, and/or fitted on, a former (not shown) having one or more appropriately shaped surfaces, the former, the frame 206 and the support members 250 are dipped into a molten leaflet material such as molten polyurethane, and the leaflet material is allowed, or caused, to solidify around the frame 206 and the support members 250 with a shape which is dictated by the one or more surfaces of the former to form the leaflets 204. Alternatively, an injection or extrusion moulding process may be used to form the leaflets 204 around the frame 206 and the support members 250.

Alternatively, the leaflets 204 may be formed on, or around, the frame 206, for example using a moulding process, and the support members 250 may be subsequently attached to the leaflets 204.

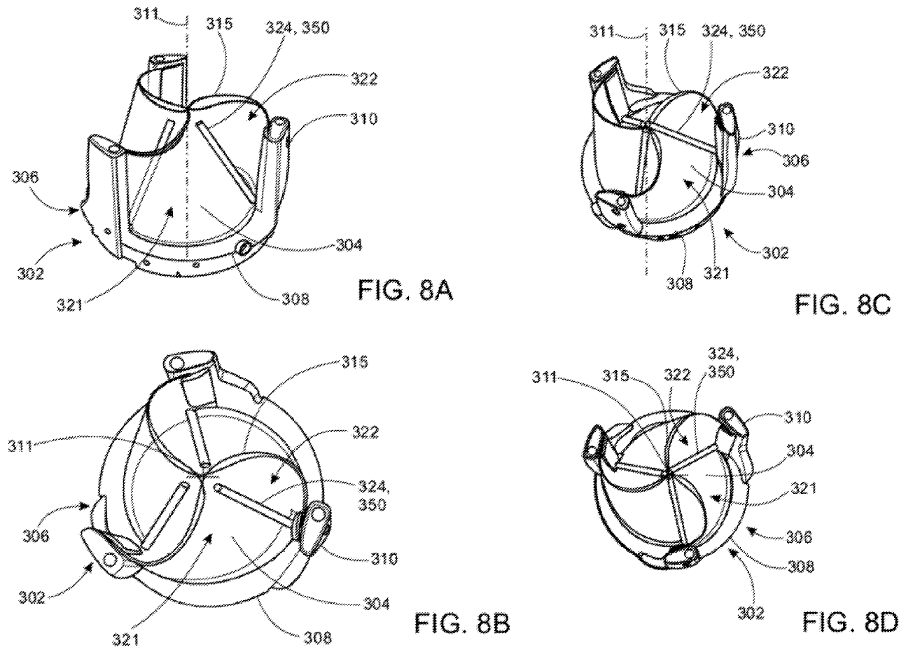

Referring now to FIGS. 8A-8D there is shown a first alternative artificial heart valve generally designated 302 for use, in particular though not exclusively in place of the aortic valve of a patient, the heart valve 302 comprising three flexible polyurethane leaflets 304 attached to a titanium frame 306. The artificial heart valve 302 of FIGS. 8A-8D includes many features which correspond closely to features of the artificial heart valve 202, wherein like features of the artificial heart valve 302 of FIGS. 8A-8D have reference numerals which are given by the reference numerals of the corresponding features of the artificial heart valve 202 incremented by “100”.

The frame 306 comprises a generally annular base 308 defining an aperture and three posts 310 extending from the base 308. The aperture defines an axis 311. The posts 310 are generally straight and extend from the base 308 in a direction which is generally parallel to the axis 311.

A base portion of each leaflet 304 is outwardly convex relative to the axis 311 and extends partially around, and is attached to, the annular base 308 of the frame 306 along a corresponding base line of attachment. Side portions of each leaflet 304 are attached to corresponding first and second adjacent posts 310 of the frame 306 along corresponding side lines of attachment. Each leaflet 304 has a free edge 315 which extends between the corresponding first and second adjacent posts 310 of the frame 306 opposite the base 308 of the frame 306. The free edge 315 of each leaflet 304 is free to move relative to the frame 306 in response to a difference in pressure between an inflow side of the leaflets 304 and an outflow side of the leaflets 304.

Each leaflet 304 comprises an outwardly convex region 321 and an outwardly concave region 322, wherein the corresponding outwardly convex and outwardly concave regions 321, 322 meet along a corresponding demarcation line 324. Moreover, as may be appreciated from the following description, the outwardly convex region 321 may comprise a region of an outer surface of a corresponding cone such as a right cone, an oblique cone or an elliptic cone. Similarly, as may be appreciated from the following description, the outwardly concave region 322 may comprise a region of an inner surface of a corresponding cone such as a right cone, an oblique cone or an elliptic cone.

The artificial heart valve 302 further comprises three linear titanium support members 350, wherein each support member 350 is attached to an outer surface of a corresponding leaflet 304 relative to the axis 311 and extends along the demarcation line 324 of the corresponding leaflet 304.

In use, the leaflets 304 are configured to move between an open configuration and a closed configuration like the leaflets 204 of the artificial heart valve 202. However, unlike the leaflets 204 of the artificial heart valve 202, the leaflets 304 of the artificial heart valve 302 of FIGS. 8A-8D do not define a defined co-aptation region like co-aptation region 217. Instead, when in the closed configuration and exposed to a back pressure, the leaflets 304 of the artificial heart valve 302 of FIGS. 8A-8D simply meet along their free edges 315 so as to form a seal therebetween which prevents, or at least partially restricts, the flow of blood in the backward direction. In other respects, the operation of the artificial heart valve 302 is essentially the same as the operation of the artificial heart valve 202.

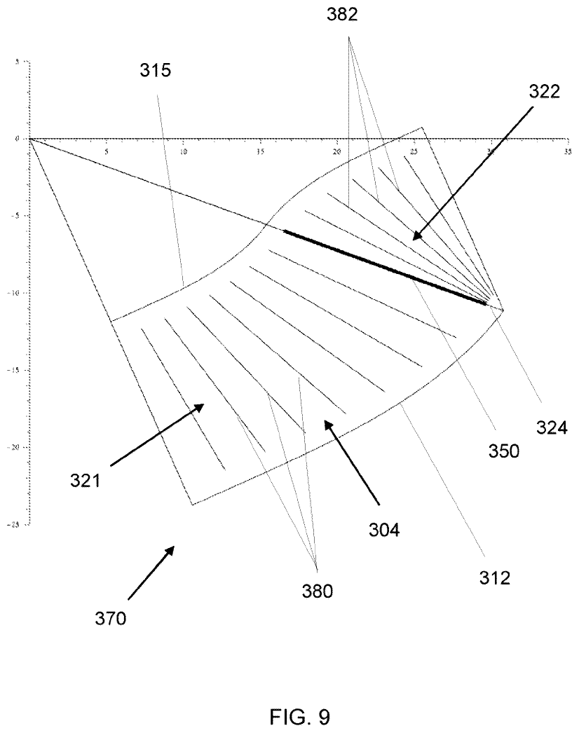

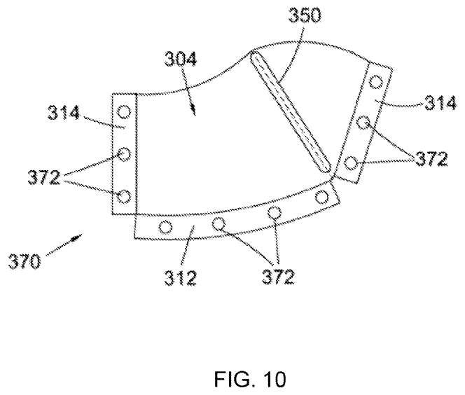

The artificial heart valve 302 mainly differs from the artificial heart valve 202 in the method used to manufacture the artificial heart valve 302. Specifically, the leaflets 304 of the artificial heart valve 302 are formed separately from the frame 306 and a corresponding support member 350 is attached to each leaflet 204, at least partially embedded within each leaflet 204, and/or at least partially inserted within each leaflet 204 so as to form a leaflet arrangement and the leaflet arrangement is subsequently attached to the frame 206. Moreover, as a consequence of the specific shape of the leaflets 304, the inventors have realised that, unlike “cusp-shaped” leaflets formed from a synthetic material such as polyurethane, each leaflet 304 may be formed separately from the frame 306 as a flat sheet and then attached to the frame 306. For example, FIGS. 9 and 10 show a generally flat leaflet arrangement generally designated 370 prior to attachment of the leaflet arrangement 370 to the frame 306. The leaflet arrangement 370 comprises a polyurethane leaflet 304 and a linear titanium support member 350 attached to an outer surface of the leaflet 304. Prior to attachment of the leaflet arrangement 370 to the frame 306, the leaflet 304 is generally flat. After attachment of the leaflet arrangement 370 to the frame 306, the leaflet 304 adopts the shape described above with reference to FIGS. 8A-8D. As such, one of skill in the art will understand that each leaflet 304 may be formed from a flat sheet of polyurethane material or may be formed initially so as to be generally flat.

FIG. 9 shows that the cones of the outwardly convex and outwardly concave regions 321, 322 are inverted relative to each other. The demarcation line 350 intersects the base edge 312 of the leaflet 304 at the vertex of the cone of the outwardly concave region 522. The vertex of the cone of the outwardly convex region 321 is the (0,0) point in the co-ordinate system shown in FIG. 9. The outwardly convex region 321 takes the form of a truncated cone, delimited by the free edge 315 of the leaflet 304. Generator lines 380 of the cone of the outwardly convex region 321 of the leaflet 304 when laid flat, and generator lines 382 of the cone of the outwardly concave region 322 of the leaflet 304 when laid flat. The demarcation line 324, at which the support member 324 is attached to the leaflet 304, is a coincident generator line of the outwardly concave and outwardly convex regions of the leaflet 304.

As shown in FIG. 10, the base portion 312 and the side portions 314 of the leaflet 304 each define a plurality of holes 372 for attachment of the leaflet arrangement 370 to the frame 306. The base 308 of the frame 306 includes a plurality of pins, pegs or protrusions (not shown) which extend radially from the base 308 of the frame 306, wherein each pin, peg or protrusion is configured to extend through a corresponding one of the holes 372 in the base portion 312 of the leaflet 304. Similarly, each post 310 of the frame 306 includes a plurality of pins, pegs or protrusions (not shown) which extend radially from the post 310 of the frame 306, wherein each pin, peg or protrusion is configured to extend through a corresponding one of the holes 372 in the side portions 314 of two of the leaflets 304.

The artificial heart valve 302 is formed by fitting the holes 372 in the base portion 312 of each leaflet 304 of each leaflet arrangement 370 over the corresponding pins, pegs or protrusions extending radially from the base 308 of the frame 306 and fitting the holes 372 in the side portions 312 of each leaflet 304 of each leaflet arrangement 370 over the corresponding pins, pegs or protrusions extending radially from the posts 310 of the frame 306. Conversely, the artificial heart valve 302 may be formed by pushing pins, pegs or protrusions extending radially from the base 308 of the frame 306 through the corresponding holes 372 in the base portion 312 of each leaflet 304 of each leaflet arrangement 370 and pushing the pins, pegs or protrusions extending radially from the posts 310 of the frame 306 through the corresponding holes 372 in the side portions 312 of each leaflet 304 of each leaflet arrangement 370. The leaflet arrangements 370 may then be secured in place on the frame 306 by mechanically clamping the leaflet arrangements 370 to the frame 306 and/or by bonding the leaflet arrangements 370 to the frame 306, for example using an adhesive.

In use, the operation of the artificial heart valve 302 is generally identical to the operation of the artificial heart valve 202 described above.

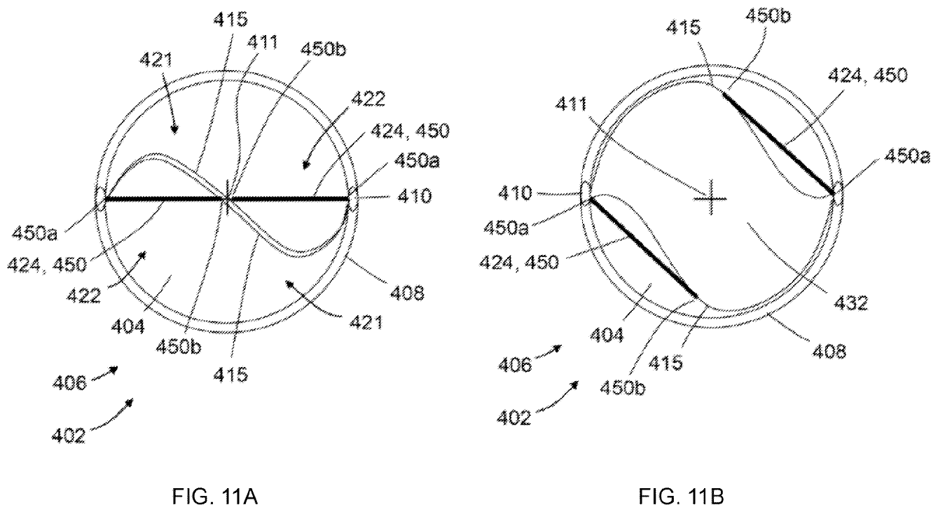

Referring now to FIGS. 11A and 11B there is shown a second alternative artificial heart valve generally designated 402 for use, in particular though not exclusively in place of the mitral valve of a patient, the heart valve 402 comprising two flexible polyurethane leaflets 404 attached to a titanium frame 406. The artificial heart valve 402 includes many features which correspond closely to features of the artificial heart valve 202, wherein like features of the artificial heart valve 402 have reference numerals which are given by the reference numerals of the corresponding features of the artificial heart valve 202 incremented by “200”.

The frame 406 comprises a generally annular base 408 defining an aperture and two posts 410 extending from the base 408. The aperture defines an axis 411. The posts 410 are generally straight and extend from the base 408 in a direction which is generally parallel to the axis 411.

A base portion of each leaflet 404 is outwardly convex relative to the axis 411 and extends partially around, and is attached to, the annular base 408 of the frame 406 along a corresponding base line of attachment. Side portions of each leaflet 404 are attached to corresponding first and second adjacent posts 410 of the frame 406 along corresponding side lines of attachment. Each leaflet 404 has a free edge 415 which extends between the corresponding first and second adjacent posts 410 of the frame 406 opposite the base 408 of the frame 406. The free edge 415 of each leaflet 404 is free to move relative to the frame 406 in response to a difference in pressure between an inflow side of the leaflets 404 and an outflow side of the leaflets 404.

Each leaflet 404 comprises an outwardly convex region 421 and an outwardly concave region 422, wherein the corresponding outwardly convex and outwardly concave regions 421, 422 meet along a corresponding demarcation line 424. Moreover, as may be appreciated from the following description, the outwardly convex region 421 may comprise a region of an outer surface of a corresponding cone such as a right cone, an oblique cone or an elliptic cone. Similarly, as may be appreciated from the following description, the outwardly concave region 422 may comprise a region of an inner surface of a corresponding cone such as a right cone, an oblique cone or an elliptic cone.

Each leaflet 404 comprises a plurality of transverse cross-sections, wherein each transverse cross-section is taken through said leaflet 404 in a corresponding transverse plane which is transverse to the axis 411 and wherein each transverse cross-section extends along a corresponding curved line. Each transverse plane extends through the outwardly convex region 421, the outwardly concave region 422, and the demarcation line 424 of said leaflet 404 so as to define a corresponding transverse cross-section comprising a corresponding outwardly convex portion, a corresponding outwardly concave portion, and a corresponding point of inflection respectively. The corresponding outwardly convex portion is outwardly convex relative to the axis 411, the corresponding outwardly concave portion 420 is outwardly concave relative to the axis 411, and the corresponding point of inflection is located at a corresponding junction between the corresponding outwardly convex portion and the corresponding outwardly concave portion so that each transverse cross-section has an “S-shape” when viewed from an outflow direction. The corresponding outwardly convex portion of each transverse cross-section extends from a corresponding first post 410 to the corresponding point of inflection, and the corresponding outwardly concave portion of each transverse cross-section extends from the corresponding second post 410 to the corresponding point of inflection.

The artificial heart valve 402 further comprises two linear titanium support members 450, wherein each support member 450 is attached to an outer surface of a corresponding leaflet 404 relative to the axis 411 and extends along the demarcation line 424 of the corresponding leaflet 404. Each support member 450 is resistant to compression along a length thereof and is more rigid than the corresponding leaflet 404. Each support member 450 comprises a base end 450a and a free end 450b, wherein the free end 450b of each support member 450 is movable with the corresponding leaflet 404 over a greater range of movement than the corresponding base end 450a. The base end 450a of each support member 450 is disposed towards the frame 406 but is only attached to the frame 406 indirectly by virtue of the attachment of the support member 450 to the corresponding leaflet 404 and attachment of the corresponding leaflet 404 to the frame 406.

In use, when the leaflets 404 are configured in the closed configuration so that the free edges 415 of the leaflets 404 extend along the paths shown in FIG. 10A and the leaflets 404 are exposed to a back pressure acting in a backward direction on an outflow side of the leaflets 404, the radially inner surfaces of the leaflets 404 adjacent the free edges 415 of the leaflets 404 engage one another so as to reduce or prevent blood flow between the leaflets 404 in the backward direction, the free ends 450b of the support members 450 support one another, and the base end 450a of each support member 450 is supported by the frame 406 so that the support members 450 together form a support structure in the form of an inverted V-shaped support structure as shown by the straight dashed lines in FIG. 10A and so that the support members 450 at least partially transfer the force exerted on the leaflets 404 to the frame 406 to thereby prevent or resist displacement of the leaflets 404 relative to the frame 406 in the backward direction. Specifically, when the leaflets 404 are configured in the closed configuration and the leaflets 404 are exposed to a back pressure acting in the backward direction, the free ends 450b of the support members 450 support one another at a position on the axis 411, and the base end 450a of each support member 450 is supported by the frame 406 so that the support members 450 together form an inverted V-shaped support structure and the support members 450 at least partially transfer the force exerted on the leaflets 404 to the frame 406 to thereby prevent or resist displacement of a middle portion of the leaflets 404 relative to the frame 406 in the backward direction.

When the pressure exerted on the flexible leaflets 404 from an inflow side of the leaflets 404 sufficiently exceeds that exerted from the outflow side, the free edges 415 of the leaflets 404 disengage, the free ends 450b of the support members 450 no longer support one another and the leaflets 404 move radially outwardly until the free edges 415 adopt an open configuration shown in FIG. 10B to create an outflow orifice 432, the maximum size of which can be varied by the design of the S-shape that determines the length of the free edges 415 of the leaflets 404.

In other respects, the structure and operation of the artificial heart valve 402 is generally similar to the structure and operation of the artificial heart valve 202 described above.

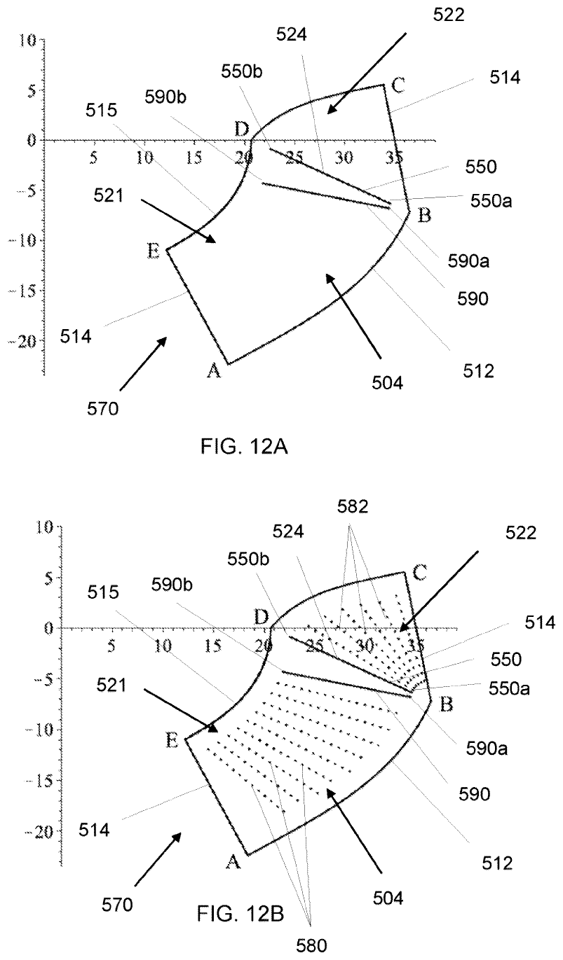

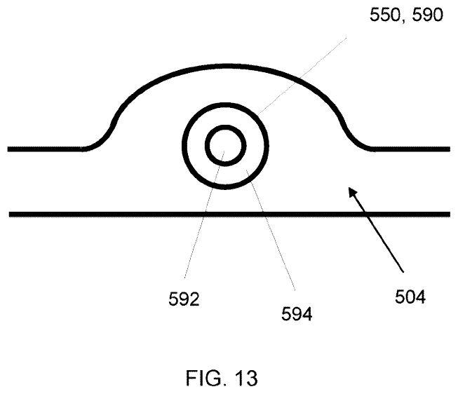

Referring now to FIGS. 12A, 12B and 13, there is shown an alternative embodiment of a leaflet arrangement 570. The leaflet arrangement 570 of FIGS. 12A, 12B and 13 includes many features which correspond closely to features of the leaflet 204 and support member 250, the leaflet arrangement 370, and the leaflet 404 and support member 450. Like features of the leaflet arrangement 570 have reference numerals which are given by the reference numerals of the corresponding features of the leaflet arrangement 370 incremented by “200”.

FIGS. 12A and 12B show a generally flat leaflet arrangement, generally designated 570, prior to attachment of the leaflet arrangement 570 to a frame. The leaflet arrangement 570 comprises a polyurethane leaflet 504 and a linear titanium support member 550 attached to an outer surface of the leaflet 504. Prior to attachment of the leaflet arrangement 570 to the frame, the leaflet 304 is generally flat. After attachment of the leaflet arrangement 570 to the frame, the leaflet 504 adopts a shape comprising an outwardly convex region 521 and an outwardly concave region 522, wherein the corresponding outwardly convex and outwardly concave regions 521, 522 meet along a corresponding demarcation line 524. The outwardly convex region 521 may comprise a region of an outer surface of a corresponding cone such as a right cone, an oblique cone or an elliptic cone. Similarly, the outwardly concave region 522 may comprise a region of an inner surface of a corresponding cone such as a right cone, an oblique cone or an elliptic cone. The leaflet 504 is configured for use in an artificial heart valve having an inner radius of 23 mm, and a height of 13 mm.

In use, the operation of an artificial heart valve comprising the leaflet arrangements 570 is generally identical to the operation of the artificial heart valve 202 described above. Leaflets 504 in an artificial heart valve are configured to move between an open configuration and a closed configuration like the leaflets 204 of the artificial heart valve 202.

The leaflet 504 comprises a free edge 515 extending between vertices E-C, a base edge 512 extending between vertices A-B, and side edges 514 extending between vertices A-E and B-C respectively. The demarcation line extends between vertex B and the free edge 515. Vertex B is otherwise referred to as the primary vertex. The demarcation line intersects the free edge at point D.

The cones of the outwardly convex and outwardly concave regions 521, 522 are inverted relative to each other. Vertex B, the primary vertex, is the vertex of the cone of the outwardly concave region 522. The vertex of the cone of the outwardly convex region 521 is the (0,0) point in the co-ordinate system shown in FIGS. 12A and 12B. The outwardly convex region 521 takes the form of a truncated cone, delimited by the free edge 515 of the leaflet 504.

The co-ordinates of the vertices A, B, C and E, and of point D, for the illustrated leaflet are provided in the table below.

| | cartesian coordinates | polar coordinates |
|---|---|---|
| Vertex/point | (mm, mm) | (mm, deg) |
| A | (18.38, −22.40) | (28.98, −50.6°) |
| B | (36.44, −7.21) | (37.15, −11.2°) |
| C | (33.84, 5.53) | (34.29, 9.3°) |
| D | (20.64, 0) | (20.64, 0°) |
| E | (12.18, −10.97) | (16.39, −42.0°) |

As shown in FIG. 12B, the cones of the outwardly convex and outwardly concave regions 521, 522 define generator lines 580, 582 which are generally straight both when the leaflet is flat and when the leaflet forms part of an artificial heart valve and takes the shape described above. In particular, the generator lines 580, 582 are straight both when the leaflet is flat and when the leaflet forms part of an artificial heart valve and is in the closed configuration.

As in the leaflet arrangement 370, the support member 550 extends along the demarcation line 524 of the corresponding leaflet 504. However, the leaflet 504 differs from the leaflet 304 in that the demarcation line is a generator line 582 of only the cone of the outwardly concave region 522, not the cone of the outwardly convex region 521. This permits the length of the free edge of the outwardly convex region 521 of the leaflet 504 to be longer, thus permitting a larger outflow orifice when the leaflet forms part of an artificial heart valve and is in the open configuration and further encouraging helical flow through the valve.

The leaflet arrangement 570 further comprises a supplementary support member 590 attached to the leaflet 504. The supplementary support member 590 is configured as per the support member 550, except for the location of the supplementary support member 590. The supplementary support member 590 is attached to the outwardly convex region 521 of the leaflet and extends along a generator line 580 of the cone of the outwardly convex region 521 of the leaflet 504. In particular, the supplementary support member 590 extends along a generator line 580 of the cone of the outwardly convex region 521 that intersects the primary vertex B.

One of ordinary skill in the art may envisage that further supplementary support members may be provided on any other generator line of either the cone of the outwardly convex region or the cone of the outwardly concave region. However, positioning the supplementary support member on a generator line proximal the demarcation line is preferred in order to support the area of transition between the outwardly convex and outwardly concave regions which may be most prone to buckling.

Free ends 550b, 590b of the support member 550 and the supplementary support member 590 are located adjacent to the free edge 515 of the leaflet 504, but offset from the free edge 515 of the leaflet 504. The offset between the free ends 550b, 590b of the support member 550 and the supplementary support member 590 and the free edge 515 of the leaflet 504 is approximately 2 mm.

Base ends 550a, 590a of the support member and the supplementary support member 590 are located adjacent the base edge 512 of the leaflet 504, in particular adjacent the primary vertex B, but spaced from the base edge 512 by a gap of approximately 2 mm.

The support member 550 and the supplementary support member 590 are both embedded in an outer surface of the leaflet 504 as shown in FIG. 13. The member 550, 590 comprises a core 592 and a coating 594. The coating 594 may allow for neutral inclusion of the member 550, 590 such that the member 550, 590 may not impact stress distributions, e.g. cause stress concentrations, in the leaflet 504. The core 592 is cylindrical. The core 592 has a thickness, e.g. diameter, less than 1 mm. The core 592 is formed of a first material, such as titanium. The coating 594 has a thickness up to 1.5 times larger than a radius of the core 592. Thus, a diameter of the member 550, 590 may be up to 2.5 times the diameter of the core 592. The coating 594 is formed of a second material having a lower stiffness than the first material. The second material is a polymeric material such as polyurethane.

One of ordinary skill in the art will also understand that various modifications are possible to any of the heart valves 202, 302, 402 described above. For example, each support member 250, 350, 450, 550 and/or supplementary support member 590 may be attached to an inner surface or an outer surface of the corresponding leaflet 204, 304, 404, 504 relative to the axis 211, 311, 411. Each support member 250, 350, 450, 550 and/or supplementary support member 590 may be at least partially inserted within the corresponding leaflet 204, 304, 404, 504. Each support member 250, 350, 450, 550 and/or supplementary support member 590 may be formed on, for example deposited on, or applied to, an inner surface and/or an outer surface of the corresponding leaflet 204, 304, 404, 504 relative to the axis 211, 311, 411.

Each support member 250, 350, 450, 550 and/or supplementary support member 590 may comprise a corresponding base portion extending adjacent to the corresponding base end 250a, 350a, 450a, 550a, 590a and a corresponding main body portion extending from the corresponding base portion to the corresponding free end 250b, 350b, 450b, 550b, 590b. The base portion of each support member 250, 350, 450, 550 and/or supplementary support member 590 may be less rigid than the corresponding main body portion. The base portion of each support member 250, 350, 450, 550 and/or supplementary support member 590 may be tapered from the corresponding main body portion to the corresponding base end 250a, 350a, 450a, 440a, 490a. The base portion of each support member 250, 350, 450, 550 and/or supplementary support member 590 may have a smaller transverse cross-section than the corresponding main body portion. The base portion of each support member 250, 350, 450, 550 and/or supplementary support member 590 may be thinner and/or narrower than the corresponding main body portion. The base portion of each support member 250, 350, 450, 550 and/or supplementary support member 590 may comprise, or be formed from, a less rigid material than a material of the corresponding main body portion. The base portion and the main body portion of each support member 250, 350, 450, 550 and/or supplementary support member 590 may have different degrees of isotropy.

Each support member 250, 350, 450, 550 and/or supplementary support member 590 may be linear.

Each support member 250, 350, 450, 550 and/or supplementary support member 590 may comprise a plurality of linear portions. For example, a first linear portion of each support member 250 may extend from the base end 250a of the support member 250 to a boundary 216 of the co-aptation region 217 of the corresponding leaflet 204 and a second linear portion of each support member 250 may extend from the boundary 216 of the co-aptation region 217 of the corresponding leaflet 204 to the free edge 215 of the corresponding leaflet 204. Each support member 250, 350, 450, 550 and/or supplementary support member 590 may be piecewise linear.

The free end 250b, 350b, 450b, 550b, 590b of each support member 250, 350, 450, 550 and/or supplementary support member 590 may coincide with the free edge 215, 315, 415, 515 of the corresponding leaflet 204, 304, 404, 50, or the free end 250b, 350b, 450b, 550b, 590b of each support member 250, 350, 450, 550 and/or supplementary support member 590 may be located adjacent to the free edge 215, 315, 415, 415 of the corresponding leaflet 204, 304, 404, 504, but offset from the free edge 215, 315, 415, 515 of the corresponding leaflet 204, 304, 404, 504 in the backward direction. The free ends 250b, 350b, 450b, 550b of the support members 250, 350, 450, 450 may engage one another when the leaflets 204, 304, 404, 504 are in the closed configuration but may be disengaged when the leaflets 204, 304, 404, 504 are in the open configuration.

The base end 250a, 350a, 450a, 550a of each support member 250, 350, 450, 550 may engage the frame 206, 306, 406 when the leaflets 204, 304, 404, 504 are in the closed configuration but are disengaged from the frame 206, 306, 406 when the leaflets 204, 304, 404, 504 are in the open configuration.

The base ends 250a, 350a, 450a, 550a of the support members 250, 350, 450, 550 may engage corresponding engagement regions of the frame 206, 306, 406 when the leaflets 204, 304, 404, 504 are in the closed configuration and are exposed to back pressure, wherein the base ends 250a, 350a, 450a, 550a of the support members 250, 350, 450, 550 and the corresponding engagement regions of the frame 206, 306, 406 are complementary in shape.

Rather than the base end 250a, 350a, 450a, 550a of each support member 250, 350, 450, 550 only being attached to the frame 206, 306, 406 indirectly by virtue of the attachment of the support member 250, 350, 450, 550 to the corresponding leaflet 204, 304, 404, 504 and attachment of the corresponding leaflet 204, 304, 404, 504 to the frame 206, 306, 406, the base end 250a, 350a, 450a, 550a of each support member 250, 350, 450, 550 may be attached to the frame 206, 306, 406 directly by a corresponding pivot joint. For example, the base end 250a, 350a, 450a, 550a of each support member 250, 350, 450, 550 and the frame 206, 306, 406 may together define a pivot joint therebetween such as a ball and socket joint or a hinge joint. Each pivot joint may be formed integrally with the frame 206, 306, 406, for example using an additive manufacturing technique such as 3D printing. When the base end 250a, 350a, 450a, 550a of each support member 250, 350, 450, 550 is pivotally attached to the frame 206, 306, 406 directly by a corresponding pivot joint, the heart valve may be manufactured by either forming the leaflets 204, 304, 404, 504 on, or around, the frame 206, 306, 406 and the support members 250, 350, 450, 550, for example using a moulding process. Alternatively, the leaflets 204, 304, 404, 504 may be formed separately from the frame 206, 306, 406 and the support members 250, 350, 450, 550, wherein each leaflet 204, 304, 404, 504 defines a socket or a pocket for receiving the free end 250b, 350b, 450b, 550b of a corresponding support member 250, 350, 450, 550, and the free end 250b, 350b, 450b, 550b of a corresponding support member 250, 350, 450, 550 may subsequently be inserted into the socket or pocket of the corresponding leaflet 204, 304, 404, 504 and secured in place, for example using an adhesive or an epoxy. It should also be understood that, in practice, where each support member 250, 350, 450, 550 is attached to the frame 206, 306, 406 directly by a corresponding pivot joint and the leaflets 204, 304, 404, 504 are in the open configuration, the base end 250a of each support member 250 may be separated from the frame 206 by a corresponding gap which is so small that, when the leaflets 204 move towards the closed configuration and are exposed to back pressure, any displacement of the leaflets 204 in the backward direction and/or any buckling of the leaflets 204 due to movement of the base end 250a of each support member 250 across the corresponding gap towards the frame 206 is insignificant.

Each support member and/or supplementary support member may comprise, or be formed from, titanium, carbon fibre, graphene, or a polymeric material such as polyurethane.

Each leaflet may comprise, or be formed from, a flexible material such as a flexible polymer material.

Each leaflet may comprise, or be formed from, polyurethane.

Each leaflet may comprise, or be formed from, a flexible composite material which includes a flexible matrix material and one or more flexible reinforcing elements embedded within the flexible matrix material, wherein the one or more flexible reinforcing elements are distinct from the corresponding supporting member and/or supplementary support member.

Each leaflet comprises, or is formed from, lower modulus polyurethane and each support member and/or supplementary support member comprises, or is formed from, higher modulus polyurethane.

The frame may comprise, or be formed from, at least one of a metal, titanium, stainless steel, a polymer or polyether ether ketone (PEEK).

Although the disclosure has been described in terms of preferred embodiments as set forth above, it should be understood that these embodiments are illustrative only and that the claims are not limited to those embodiments. Those skilled in the art will be able to make modifications and alternatives to the described embodiments in view of the disclosure which are contemplated as falling within the scope of the appended claims. Each feature disclosed or illustrated in the present specification may be incorporated in any embodiment, whether alone or in any appropriate combination with any other feature disclosed or illustrated herein. In particular, one of ordinary skill in the art will understand that one or more of the features of the embodiments of the present disclosure described above with reference to the drawings may produce effects or provide advantages when used in isolation from one or more of the other features of the embodiments of the present disclosure and that different combinations of the features are possible other than the specific combinations of the features of the embodiments of the present disclosure described above.

The skilled person will understand that in the preceding description and appended claims, positional terms such as ‘above’, ‘along’, ‘side’, etc. are made with reference to conceptual illustrations, such as those shown in the appended drawings. These terms are used for ease of reference but are not intended to be of limiting nature. These terms are therefore to be understood as referring to an object when in an orientation as shown in the accompanying drawings.

Use of the term “comprising” when used in relation to a feature of an embodiment of the present disclosure does not exclude other features or steps. Use of the term “a” or “an” when used in relation to a feature of an embodiment of the present disclosure does not exclude the possibility that the embodiment may include a plurality of such features.

The use of reference signs in the claims should not be construed as limiting the scope of the claims.

1. An artificial heart valve comprising:
a frame;
a plurality of flexible leaflets attached to the frame; and
a plurality of support members, each support member being attached to a corresponding leaflet,
wherein the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein the support members are movable with the leaflets relative to the frame, and
wherein, when the leaflets are in the closed configuration and exposed to a back pressure having at least a component in the backward direction, a force is exerted on the leaflets in the backward direction, and the support members at least partially prevent or resist deformation of the leaflets.

2. The artificial heart valve as claimed in claim 1, wherein each leaflet comprises an outwardly convex region, an outwardly concave region, and a demarcation line, wherein the outwardly convex and outwardly concave regions meet along the demarcation line.

3. The artificial heart valve as claimed in claim 2, wherein at least one of:
the artificial heart valve is configured such that movement of the leaflets results in a change in a curvature of the outwardly convex and outwardly concave regions of each leaflet and a change in orientation of the corresponding demarcation line, but no change in shape of the corresponding demarcation line;
the demarcation line of each leaflet is linear;
the demarcation line of each leaflet comprises a plurality of linear portions;
the demarcation line of each leaflet is piecewise linear; or
each support member extends at least part way along the demarcation line of the corresponding leaflet.

4. (canceled)

5. (canceled)

6. The artificial heart valve as claimed in claim 1, further comprising a plurality of supplementary support members, at least one supplementary support member being attached to a corresponding leaflet.

7. The artificial heart valve of claim 6, wherein each leaflet comprises an outwardly convex region, an outwardly concave region, and a demarcation line, wherein the outwardly convex and outwardly concave regions meet along the demarcation line, and wherein the at least one supplementary support member is attached to the outwardly convex region of the corresponding leaflet.

8. The artificial heart valve as claimed in claim 1, wherein at least one of:
each support member is attached to an inner surface or an outer surface of the corresponding leaflet;
each support member is at least partially embedded within the corresponding leaflet;
each support member is at least partially inserted within the corresponding leaflet;
each leaflet comprises a socket or a pocket for receiving at least part of the corresponding support member;
each support member is formed on, for example deposited on, or applied to, an inner surface and/or an outer surface of the corresponding leaflet.

9. The artificial heart valve as claimed in claim 1, wherein at least one of:
each support member is elongated;
each support member is linear;
each support member comprises a plurality of linear portions;
each support member is piecewise linear;
each support member is resistant to compression along a length thereof;
each support member is more rigid than the corresponding leaflet.

10. The artificial heart valve as claimed in claim 1, wherein at least one of:
each support member comprises a base end and a free end, and wherein the free end of each support member is movable with the corresponding leaflet over a greater range of movement than the corresponding base end, wherein the base end of each support member is disposed towards the frame, and wherein the free end of each support member coincides with the free edge of the corresponding leaflet, or the free end of each support member is located adjacent to the free edge of the corresponding leaflet but offset from the free edge of the corresponding leaflet in the backward direction;
the free ends of the support members support one another when the leaflets are in the closed configuration but do not support one another when the leaflets are in the open configuration;
the free ends of the support members engage one another when the leaflets are in the closed configuration but are disengaged when the leaflets are in the open configuration;
the free ends of the support members are complementary in shape to one another;
the base end of each support member is supported by the frame when the leaflets are in the closed configuration but is not supported by the frame when the leaflets are in the open configuration; or
the base end of each support member engages the frame when the leaflets are in the closed configuration but is disengaged from the frame when the leaflets are in the open configuration.

11. (canceled)

12. (canceled)

13. The artificial heart valve as claimed in claim 10, wherein at least one of:
the base end of each support member is only attached to the frame indirectly by virtue of the attachment of the support member to the corresponding leaflet and attachment of the corresponding leaflet to the frame; or
the base ends of the support members engage corresponding engagement regions of the frame when the leaflets are in the closed configuration and the leaflets are exposed to the back pressure, wherein the base ends of the support members and the corresponding engagement regions of the frame are complementary in shape.

14. (canceled)

15. The artificial heart valve as claimed in claim 10, wherein at least one of:
the base end of each support member is pivotally attached to the frame directly by a corresponding pivot joint, for example wherein the base end of each support member and the frame may define a pivot joint therebetween such as a ball and socket joint or a hinge joint;
the base end of each support member is spaced from the frame by a gap of between 1 mm and 5 mm; or
each support member comprises a corresponding base portion extending adjacent to the corresponding base end and a corresponding main body portion extending from the corresponding base portion to the corresponding free end and wherein the base portion of each support member is less rigid than the corresponding main body portion.

16. (canceled)

17. (canceled)

18. The artificial heart valve as claimed in claim 2, wherein, when viewed from an outflow side of the heart valve looking towards the free edges of the leaflets, the outwardly convex region of each leaflet precedes the corresponding outwardly concave region of said leaflet in a counter-clockwise direction defined around the axis.

19. The artificial heart valve as claimed in claim 2, wherein the frame comprises a base defining an aperture for blood flow and a plurality of posts extending from the base, wherein each leaflet comprises a corresponding base portion attached to the base of the frame along a base line of attachment extending around a corresponding portion of the aperture, wherein the base line of attachment is outwardly convex relative to an axis defined by the aperture, wherein each leaflet comprises first and second side portions, and wherein the first and second sides portions of each leaflet are attached to corresponding first and second adjacent posts along first and second at least partially straight lines of attachment respectively.

20. The artificial heart valve as claimed in claim 19, wherein each leaflet comprises a plurality of transverse cross-sections, wherein each transverse cross-section is taken through said leaflet in a corresponding transverse plane which is transverse to an axis defined by the aperture, and
wherein each transverse plane extends through the outwardly convex region, the outwardly concave region, and the demarcation line of said leaflet so as to define a corresponding transverse cross-section which extends along a corresponding curved line and which comprises a corresponding outwardly convex portion, a corresponding outwardly concave portion, and a corresponding point of inflection respectively, wherein the corresponding outwardly convex portion is outwardly convex relative to the axis, the corresponding outwardly concave portion is outwardly concave relative to the axis, and the corresponding point of inflection is located at a corresponding junction between the corresponding outwardly convex portion and the corresponding outwardly concave portion, and wherein the corresponding outwardly convex portion extends from the corresponding first post to the corresponding point of inflection, and the corresponding outwardly concave portion extends from the corresponding second post to the corresponding point of inflection.

21. The artificial heart valve as claimed in claim 20, wherein:
a first one of the transverse cross-sections taken through any one of the leaflets on a first one of the transverse planes comprises a first outwardly convex portion, a first outwardly concave portion, and a first point of inflection,
a second one of the transverse cross-sections taken through any one of the leaflets on a second one of the transverse planes comprises a second outwardly convex portion, a second outwardly concave portion, and a second point of inflection,
the first one of the transverse planes is located closer to the base of the frame than the second one of the transverse planes, and
the first outwardly convex portion is longer than the second outwardly convex portion and the first outwardly concave portion is shorter than the second outwardly concave portion.

22. The artificial heart valve as claimed in claim 20, wherein each leaflet comprises a corresponding free edge which is movable relative to the frame, wherein the corresponding free edge extends along a corresponding curved line in a transverse plane transverse to an axis defined by the aperture, and the corresponding free edge comprises a corresponding outwardly convex portion, a corresponding outwardly concave portion and a corresponding point of inflection between the corresponding outwardly convex portion and the corresponding outwardly concave portion, wherein the corresponding outwardly convex portion of the free edge extends from the corresponding first post to the corresponding point of inflection of the free edge, and the corresponding outwardly concave portion of the free edge extends from the corresponding second post to the corresponding point of inflection of the free edge, and wherein the point of inflection of the free edge is located half-way along, or substantially half-way along, the free edge between the corresponding first and second adjacent posts.

23. The artificial heart valve as claimed in claim 22, wherein the free edge of any leaflet is longer than the base portion of said leaflet and the free edge of any leaflet is longer than any of the transverse cross-sections of said leaflet.

24. The artificial heart valve as claimed in claim 1, wherein the support member comprises a core and a coating, wherein the coating has a thickness equal to or larger than a thickness of the core, wherein the core is formed of a first material and the coating is formed of a second material, and wherein the second material has a lower stiffness than the first material.

25. A leaflet arrangement for an artificial heart valve, the leaflet arrangement comprising:
a flexible leaflet configured for attachment to a frame of the artificial heart valve; and
a support member attached to the leaflet,
wherein, the leaflet arrangement is attachable to the frame together with one or more other identical leaflet arrangements, so that:
the leaflets of the leaflet arrangements are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
the support members of the leaflet arrangements are movable with the leaflets relative to the frame, and
when the leaflets of the leaflet arrangements are in the closed configuration and exposed to a back pressure having at least a component in the backward direction, a force is exerted on the leaflets in the backward direction and the support members at least partially prevent or resist deformation of the leaflets.

26. The leaflet arrangement as claimed in claim 25, wherein at least one of:
the leaflet arrangement is flat or generally flat;
the support member is attached to an inner surface or an outer surface of the leaflet;
the support member is at least partially embedded within the leaflet;
the support member is at least partially inserted within the leaflet;
the leaflet comprises a socket or a pocket for receiving a free end of the support member, and the free end of the support member is inserted into the socket or pocket;
the support member is formed separately from the leaflet;
the support member comprises, or is formed from, a different material to the leaflet;
the support member is formed on, for example deposited on, or applied to, an inner surface and/or an outer surface of the leaflet.

27. The leaflet arrangement as claimed in claim 25- or 26, further comprising a supplementary support member attached to the leaflet.

28. A method of manufacturing an artificial heart valve, the method comprising:
attaching a corresponding support member to each flexible leaflet of a plurality of flexible leaflets, and
attaching each leaflet to a frame,
wherein the leaflets are movable relative to the frame between an open configuration, in which the leaflets permit blood flow therebetween in a direction having at least a component in a forward direction, and a closed configuration, in which the leaflets prevent or restrict blood flow therebetween in a direction having at least a component in a backward direction which is opposite to the forward direction,
wherein the support members are movable with the leaflets relative to the frame, and
wherein, when the leaflets are in the closed configuration and exposed to a back pressure having at least a component in the backward direction, a force is exerted on the leaflets in the backward direction and the support members at least partially prevent or resist deformation of the leaflets.

29. The method as claimed in claim 28, comprising at least one of:
forming each leaflet separately from the frame and then attaching each leaflet to the frame;
forming each leaflet on the frame;
forming each leaflet on the frame by moulding;
forming each leaflet as a flat sheet;
forming each leaflet from a corresponding flat sheet of flexible material, for example by cutting the corresponding flat sheet of flexible material;
attaching each support member to an inner surface or an outer surface of the corresponding leaflet;
at least partially embedding each support member within the corresponding leaflet;
at least partially inserting each support member within the corresponding leaflet;
forming each support member separately from the corresponding leaflet;
forming each support member on an inner surface and/or an outer surface of the corresponding leaflet;
forming each support member on an inner surface and/or an outer surface of the corresponding leaflet by depositing each support member on the inner surface and/or the outer surface of the corresponding leaflet;
applying each support member to an inner surface and/or an outer surface of the corresponding leaflet; or
attaching a corresponding supplementary support member to each flexible leaflet of a plurality of flexible leaflets.

30. (canceled)

31. (canceled)
